(19) United States
(12) Patent Application Publication
(10) Pub. No.: US 2002/0192711 A1
(43) Pub. Date: Dec. 19, 2002

(54) Methods for identifying ligands of G-Protein-Coupled receptors

(76) Inventors: John J. Nestor, Bedford, MA (US); Carol J. Wilson, Somerville, MA (US); Lewis C. Cantley, Cambridge, MA (US); Michael B. Yaffe, Jamaica Plain, MA (US); Ailan Guo, Medford, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/126,430

(22) Filed: Apr. 19, 2002

Related U.S. Application Data
(60) Provisional application No. 60/285,380, filed on Apr. 20, 2001.
(60) Provisional application No. 60/350,712, filed on Nov. 12, 2001.

(51) Int. Cl. C12Q 1/68; G01N 33/53; G01N 33/543; A61K 31/00

(52) U.S. Cl. 435/7.1; 435/6; 436/518; 514/1

(57) ABSTRACT

Methods for identifying ligands of G-protein coupled receptors are provided. Methods generally involve the steps of associating a GPCR having a functional conformation with a support, interacting a naturally-derived sample with the GPCR to bind a molecule in the sample to the GPCR and separating the molecule from the support.

### CROSS-REFERENCE TO RELATED APPLICATIONS

This application claims the benefit of and priority to U.S. Ser. No. 60/285,380, filed Apr. 20, 2001, and claims the benefit of and priority to U.S. Ser. No. 60/350,712, filed Nov. 12, 2001. The disclosures of both of these provisional patent applications are incorporated by reference herein.

### TECHNICAL FIELD

The invention relates generally methods for identifying molecules that bind to G-Protein-Coupled Receptors as well as the identified molecules. More particularly, the invention relates to methods for identifying natural ligands that bind to orphan G-Protein-Coupled Receptors. Identified molecules can be used to treat diseases directly or can be used to design or screen for other therapeutics, such as natural or non-natural agonists or antagonists of G-Protein-Coupled Receptors. Methods according to the invention also are useful in the identification of a function of an orphan G-Protein-Coupled Receptor.

### BACKGROUND OF THE INVENTION

Receptors, in general, are molecular structures located in the cell membrane or within a cell that typically form a non-covalent, reversible bond with an extracellular agent such as an antigen, hormone or neurotransmitter. Each receptor typically binds with a specific agent or agents. A specific family of receptors is the seven transmembrane (“7TM”), G-Protein-Coupled Receptor (“GPCR”). These receptors link with a Guanine Nucleotide-Binding Protein (“G-protein”) in order to pass on a signal from the extracellular agent with which the receptor has bound. When the G-protein is bound to Guanine Diphosphate (“GDP”), the G-protein is inactive, or in an “off position,” while, when the G-protein is bound to Guanine Triphosphate (“GTP”), the G-protein is active, or in an “on position.” When the G-protein is in the on position, activation of a biological response in a cell is mediated.

A number of therapeutically-significant events in many organisms, including humans, utilize protein-mediated transmembrane signaling via GPCRs. In general, the activity of many cells in the body is regulated, at least in part, by extracellular signals. The majority of signals are transmitted into the cell interior by GPCRs. Transmission of the signal is accomplished when a ligand binds to a GPCR and a G-protein in the cell is activated. Specific GPCRs are involved in certain cell functions and certain transmission pathways.

There are varying aspects of this signaling process involving, for example, multiple receptor subtypes for GPCRs, G-protein counterparts, and a variety of intracellular secondary messengers. Signal transduction typically results in an overall or partial activation (or inactivation) of an intracellular process or processes, which depend upon the proteins that are involved. For example, important signaling molecules or neurotransmitters that bind to GPCRs include, but are not limited to, morphine, dopamine, histamine, 5-hydroxytrytamine, and adenosine.

Generally, GPCRs constitute a superfamily of proteins. There are currently over 1000 GPCRs reported in literature, which are divided into six classes: Class A (rhodopsin-like receptors, including, but not limited to, β-adrenergic and chemokin receptors); Class B (secretin-like receptors, including, but not limited to, calcitonin, secretin, and hormon); Class C (metabotropic glutamate/pheromone receptors); Class D (fungal pheromone receptors); Class E (cAMP receptors, including, but not limited to, Dictostelium); and Class Z (Bacteriorhodpsins.) See e.g., Ji TH, et. al., J. Biol. Chem. 273(28): 17299-302 (1998). The reported GPCRs include both characterized receptors and orphan receptors for which ligands have not yet been identified. See e.g., Wilson S, et. al., G protein-coupled receptors. Haga T, Bernstein G, eds. CRC press, Boca Raton, pp. 97-116 (1999); Wilson S, et. al., Br. J. Pharmacol. 125(7):1387-92 (1998); and Marchese A, et. al., Trends Pharmacol. Sci. 20(9):370-375 (1999).

Despite the large number of GPCRs, GPCRs generally share a similar tertiary molecular structure. Each GPCR comprises a string of amino acid residues of various lengths. Also, GPCRs lie within the cell membrane in seven distinct coils or transmembrane helices. The amino terminus of the GPCR lies outside the cell with the extracellular loops, while the carboxy-terminus lies inside the cell with the intracellular loops. In general, the similarity of tertiary structure is shared by the G-proteins as well. Typically, G-proteins, also referred to as heterotrimeric G-proteins, are composed of three subunits, the alpha, beta and gamma. In a typical G protein, the alpha subunit comprises two domains, a GTPase domain and an alpha-helical domain. The GTPase domain comprises helices that surround a beta sheet. The alpha-helical domain is unique to the G-proteins and comprises a long central helix surrounded by five shorter helices. The beta and gamma subunits are often referred to as the beta-gamma dimer. The beta subunit is a “beta propeller” protein comprising sheets arranged like blades on a propeller, an alpha helix and a loop that connects the helix with the “propeller blades”. The gamma subunit, on the other hand, generally, does not have an intrinsic tertiary structure; instead, it is believed to rely on the beta subunit for structural support. Interactions with the beta subunit are believed to be mediated in part by a coiled-coil interaction between the N terminal helices of the respective subunits.

Ligands for GPCRs generally include small molecules, peptides and proteins. The interactions between these ligands and their receptors vary from system to system, but they all can require the interaction with residues in several of the four extracellular domains and the N-terminus. Certain GPCRs with known ligands have been associated with many diseases and disorders including multiple sclerosis, diabetes, rheumatoid arthritis, asthma, allergies, inflammatory bowel disease, cancers, thyroid disorders, heart disease, retinitis pigmentosa, obesity, neurological disorders, osteoporosis, Human Immunodeficiency Virus (“HIV”) infection and Acquired Immune Deficiency Syndrome (“AIDS”). See e.g., Murphy P H, et. al., Pharm. Rev. 52(1):145-176 (2000); Mannstadt M, et. al., Am. J. Physiol. 277(5):F665-75 (1999); Berger E A, et. al., Ann. Rev. Immunol. 17:657-700 (1999); Saunders J, et. al., Drug Discov. Today 4(2):80-92 (1999); Hebert T E, et. al., Biochem. Cell. Biol. 76(1):1-11 (1998); Jacobson E D, et. al., Dig. Dis. 15(4-5):207-42 (1997); Meij J T, Mol. Cell. Biochem. 157(1-2):31-8 (1996); and Chanmers J, et. al., Nature 400:261-4 (1999). When categorizing GPCRs, the receptors can be divided into different classes based on the type of ligands bound. For example, such classes include, peptide, biogenic amine, nucleotide-related, lipid-based, amino acid-based, and retinal (i.e., light-based).

Among the over-1000 estimated GPCRs in the human genome, many of these (an estimated 200-500) GPCRs are referred to as “orphan” GPCRs. Several approaches have been used heretofore to identify natural ligands to these orphan GPCRs. These approaches include, for example, using known ligands in an assay, using the sequence homology of the orphan and comparing it to known GPCR sequences, assaying against arrayed families of known ligands, and using extracts in high-throughput screening with receptor-transfected mammalian cells and a general signaling assay. Many of these methods use whole cell assays to identify a mixture of materials that induces a signal. These whole cell based methods generally require the identification of a mixture of materials with the signaling activity. After the identification of a mixture of materials with the ability to stimulate signaling, steps require further purification and re-screening procedures that are repeated until the compound is eventually isolated and identified.

These approaches are also limited by the fact that the whole cell assays are prone to significant technical problems affecting signal detection resulting from natural GPCRs present in the selected receptor-transfected mammalian cell. For example, HEK-293 is known to have at least 15 GPCRs from multiple classes, with many families and even more subfamilies. As a result, these approaches are quite laborious, slow, and have been shown to have limited success. In addition, many of these approaches allow only for the detection and identification of agonists, and fail to provide approaches to detect and identify antagonists or inverse agonists, for example. In fact, these approaches have identified only a few ligands for orphan GPCRs over the past several years. See e.g., Howard A D, et al., TIPS 22(3):132-140 (2001).

Accordingly, there is a need in the art for efficient methods of identifying natural ligands for GPCRs, validating GPCRs as a target, and identifying GPCR binding therapeutics to prevent or treat disease and disorders. Also, there is a need in the art for methods to detect any ligand that binds to the GPCRs, whether a ligand is an agonist, antagonist, inverse agonist, or any molecule with a novel function.

### SUMMARY OF THE INVENTION

The present invention generally provides methods of identifying ligands for GPCRs, methods for identifying the function of GPCRs, and ligands for GPCRs. These GPCRs include orphan GPCRs, and their ligands. The invention contemplates natural ligands as well as non-natural ligands. The methods according to the invention are applicable to any GPCR, but are especially beneficial for orphan receptors.

Methods of the invention utilize purified or partially purified GPCR as the agent for carrying out the selection and isolation of natural ligands that bind to a GPCR. The isolated natural ligand can itself be used as a therapeutic or it can otherwise be used for screening for or development of agonists or antagonists. In addition, methods of the invention include solubilizing and immobilizing GPCRs to facilitate efficient ligand selection. Generally, solubilization or isolation conditions according to the invention provide a functional conformation of a GPCR and allow for identification of a ligand that binds to a GPCR of interest.

A naturally derived sample is interacted with a GPCR of interest, such as an orphan GPCR, to bind a molecule in the sample to the GPCR. This step can be performed in solution, or with a GPCR immobilized on a support. For example, a GPCR which has been tagged with a binding tag is immobilized by the tag to a support such as an affinity resin. Generally, methods according to the invention can be used with any support known to those skilled in the art. Additionally, other forms of sequestration can be used to perform the affinity purification of select ligands from extracts or fractions.

In some embodiments, the GPCR binds to a ligand in a sample in the absence of a lipid or in the absence of a lipid layer. In other embodiments, the tagged receptor can be bound to specific affinity membranes. Samples to be tested can then be incubated with the membrane and easily washed to remove non-specific binding components. Size exclusion methodology can be used to separate a purified receptor bound ligand complex from unbound components after pre-incubating the receptor with the sample. Additionally, a micellar complex containing the receptor (which can incorporate lipids as well as detergent) can be separated after binding select affinity components from a sample by differential centrifugation. Generally, the high affinity ligand can be released using low pH or high salt conditions and the structure identified by sequencing or mass spectrometry.

Methods according to the invention also allow for the screening and isolation of ligands for other proteins, such as receptors other than GPCRs. For example, proteins that are amenable to the methods provided herein include, but are not limited to, single transmembrane, PIG-tailed receptors, progesterone receptors, arresting, nuclear receptors, cytokine receptors, ion channels, receptor kinases and essentially any protein- or peptide-binding protein. PIG-tailed receptors include cathepsin D (25) and natural killer cell receptors such as CD48 and CD55 (26). See e.g., Ogier-Denis E, et. al, Biochem. Biophys. Res. Comm. 211(3):935-42 (1995); and Schubert J, et. al, Blood 76(6):1181-7 (1990). The progesterone receptor is a well-known nuclear receptor with biological relevance. See e.g., Chauchereau A, et. al., J. Biol. Chem. 275(12):8540-8548 (2000); Fewings P E, et. al., J. Neurosurg. 92(3):401-405 (2000). Arrestins are a family of soluble protein-binding proteins which are implicated in a wide range of diseases because of their role in signal termination. See e.g., Wilson C J, et. al., Curr. Biol. 3(10):683-6 (1993). These are a few examples of protein families that will have orphans from the genome that can be adapted to this format. The methods according to the invention are applicable to any GPCR (and other proteins) but is especially beneficial for orphan receptors (those with unknown function or ligands).

Once a ligand is identified, the function the GPCR of interest can be identified. Additionally, the pathway in which the ligand is involved can be identified. Furthermore, the natural ligand can be used to screen for an inhibitor of the receptor. These screens can include assays which use the receptor with the ligand, radiolabeled, along with libraries of inhibitors to look for inhibition of binding of the natural ligand. Additionally, the function of the ligand can be assayed in functional assays. Ligands isolated according to the invention can be used directly as a therapeutic treatment of a disease or disorder. Not only do ligands identified by the invention include these peptides, peptidomimetics, small molecules, or other molecules identified or isolated from the methods of the invention, but also include those that are designed based on isolated ligands. All of these molecules can be further tested for activity in the prevention or treatment of diseases and disorders by promoting or inhibiting binding to GPCRs.

Moreover, methods of the invention involve the identification of any ligand that binds to a GPCR, such as an orphan GPCR, whether the ligand is an agonist, an inverse agonist, an antagonist, or a binding protein with an unidentified function. Also, the invention includes the design and identification of binding therapeutics, namely, therapeutic peptides, proteins, peptidomimetics, or small molecules suitable for use in the prevention or treatment of diseases and disorders. Furthermore, methods of the invention also provide for the identification of additional potential targets for the prevention and treatment of diseases and disorders.

One aspect of the invention is a method for identifying a molecule capable of binding to a G-protein coupled receptor (GPCR). The method includes the steps of associating a GPCR having a functional conformation with a support, interacting a naturally-derived sample with the GPCR to bind a molecule in the sample to the GPCR, and separating the molecule from the support. In some embodiments, the GPCR is substantially free from association with a lipid layer.

This aspect of the invention can include any or all of the following features or characteristics. The method can further include the step of identifying the molecule. The naturally-derived sample can be a tissue extract. The naturally-derived sample can be a set of at least two proteins encoded by a cDNA library. Such a cDNA library can be derived from a tissue or from at least one cell isolated from a multi-cellular organism. The method can further include the steps of associating the GPCR with a second support; interacting a subset of the proteins from the set of the at least two proteins with the GPCR to bind the molecule to the GPCR; and separating the molecule from the second support. The naturally-derived sample can be selected from the group consisting of a tissue extract, a fraction from a tissue extract, a cell culture medium, an extract from a cell grown in a tissue culture, and a fraction from an extract from a cell grown in a tissue culture. This aspect includes a molecule identified by the method set forth above. The method can further include the step of determining the function of the molecule, and the molecule can include a protein. This aspect can further include a compound derived from the molecule identified by the method set forth above. The invention also can further include the step of manufacturing a compound derived from the molecule identified by the method set forth above.

Another aspect of the invention is a method for identifying a molecule capable of binding to a G-protein coupled receptor (GPCR). The method includes the steps of associating a GPCR having a functional conformation with a support; interacting a naturally-derived first set of molecules with the GPCR; interacting a second set of proteins with the GPCR; and separating the first molecule from the support. The first set can include a first molecule capable of binding to the GPCR, and the second set can include a subset of the first set and include the first molecule. This aspect of the invention can include any or all of the following or preceding features or characteristics. The first molecule can be a protein.

Another aspect of the invention is a method for identifying a molecule capable of binding to a G-protein coupled receptor (GPCR). The method includes the steps of identifying a GPCR having an undefined function or an undefined natural binding compound, selecting a naturally-derived test sample, associating the GPCR in a functional conformation with a support, interacting the naturally-derived test sample with the GPCR to bind a molecule in the sample to the GPCR, and separating the molecule from the support.

This aspect of the invention can include any or all of the following or preceding features or characteristics. The naturally-derived test sample can include a set of at least two proteins encoded by a cDNA library. The method can further include the steps of associating the GPCR with a second support; interacting a subset of the proteins from the set of the at least two proteins with the GPCR to bind the molecule to the GPCR; and separating the molecule from the second support. The naturally-derived test sample can be selected from the group consisting of a tissue extract, a fraction from a tissue extract, a cell culture medium, an extract from a cell grown in a tissue culture, and a fraction from an extract from a cell grown in a tissue culture. This aspect includes a molecule identified by the method set forth above. The method can further include the step of determining the function of the molecule. This aspect can further include a compound derived from the molecule identified by the method set forth above. The invention also can further include the step of manufacturing a compound derived from the molecule identified by the method set forth above.

A detailed description of certain embodiments of the invention is provided below. Other embodiments of the invention are apparent upon review of the detailed description that follows.

### BRIEF DESCRIPTION OF THE DRAWINGS

FIG. 1 is a schematic diagram of a generalized method according to the invention.

FIG. 2A is a graph depicting the characterization of a CCR5 receptor demonstrating the functionality of the solubilized, immobilized receptor compared directly to the activity of the receptor in membranes.

FIG. 2B is a graph depicting the characterization of an adenosine receptor demonstrating the functionality of the solubilized adenosine receptor compared directly to the activity of the adenosine receptor in membranes.

FIG. 2C is a graph comparing the activity for the M1 Muscarinic receptor demonstrating the functionality of the solubilized M1 Muscarinic receptor to the M1 Muscarinic receptor in membranes.

FIG. 2D is a graph comparing the activity for the β1-adrenergic receptor demonstrating the functionality of the solubilized β1-adrenergic receptor to the β1-adrenergic receptor in membranes.

FIG. 2E is a graph comparing the activity for the C5a receptor demonstrating the functionality of the solubilized C5a receptor to the C5a receptor in membranes.

FIG. 2F is a graph comparing the activity for the adenosine receptor demonstrating the functionality of the solubilized adenosine receptor to the adenosine receptor in membranes.

FIG. 3 is a schematic diagram outlining methods for determining the tissue, cellular, or cDNA source for use in de-orphaning a GPCR.

FIGS. 4A-4D are tables comparing the sequence homology of orphan GPCRs to known GPCRs.

FIG. 5 is a western blot gel demonstrating the isolation and identification of RANTES from a cDNA library pool using CCR5 and the de-orphaning methods of the present invention.

FIG. 6 is a chart and a schematic diagram demonstrating the isolation and identification of RANTES from tissue culture fractions using CCR5 and methods of the invention.

FIG. 7 is a western blot gel demonstrating the isolation of a natural ligand from a ligand mixture for CCR5.

FIG. 8 is a chart and schematic diagram demonstrating the isolation and identification of ligands from tissue culture fractions using C5L2 receptor and methods of the invention.

### DETAILED DESCRIPTION OF THE INVENTION

Methods of the invention provide for the identification and isolation of a ligand, such as a natural ligand, for a receptor such as a GPCR. These GPCRs include orphan GPCRs. Generally, the isolation and identification of a ligand can identify therapeutic lead compounds. Such lead compounds can be the ligand itself or a molecule having a design based on the ligand. Accordingly, these lead compounds can be, for example, natural or non-natural agonists or antagonists of the receptor. Such therapeutic lead compounds can be used for the treatment and prevention of various diseases and disorders.

As used herein, the term “natural ligand” includes any molecule, such as, a protein, a peptide, or a small molecule, that originates from, is located in, or is produced by a nucleic acid, a virus, a bacterium, a cell, a cell line, a tissue, a tissue culture, or an organism that binds to a receptor, such as a GPCR. Examples of natural ligands include, but are not limited to, molecules in an inactive, precipitated protein preparation, a translated nucleic acid (such as a translated cDNA), molecules found on or in a cell, molecules found in a whole cell preparation, molecules found in a cell membrane preparation, molecules found in a cell culture medium, molecules found on or in a cell line, molecules found in a tissue, molecules found in a tissue extract and fractions thereof, molecules found in an extract of cells grown in tissue culture and fractions thereof, and molecules found on or in an organism. Natural ligands can be extracellular or intracellular and can include protein-protein binding domains. A natural ligand can be a receptor itself. In some instances a natural ligand can bind to itself.

Also, as used herein, the term “naturally-derived” includes any material that originates from, occurs in, or is produced by a nucleic acid, a virus, a bacterium, a cell, a cell line, a tissue, a tissue culture, or an organism (such as vertebrates such as mammals). The nucleic acid, virus, bacterium, cell, cell line, tissue, tissue culture, or organism can be unaltered or can be altered, modified or treated for experimental purposes. Examples of a naturally-derived sample include, but are not limited to, an inactive, precipitated protein preparation, a nucleic acid (such as a cDNA), a translated nucleic acid, a cell, a whole cell preparation, a cell membrane preparation, a cell culture medium, a cell line, a tissue, a tissue extract and fractions thereof, an extract of cells grown in tissue culture and fractions thereof, and a sample from an organism.

Also, as used herein, the term “orphan GPCR” refers to a GPCR for which a natural ligand has not been identified or for which a known natural ligand has not yet matched with the GPCR. Orphan GPCRs include, but are not limited to, GPR30, GPR31, GPR32, GPR38, GPR39, GPR55, GPR65, GPR84, GPR92, C5L2, HM74, VSHK1, and FKSG80. Orphan GPCR, as used in the application, also refers to a GPCR whose function in nature or in vitro has not been identified or determined (in whole or in part). Accordingly, a GPCR which has no known function or which has a known function but also has a later-determined (and previously unknown or unidentified) function is an orphan receptor. Additionally, even though a GPCR may have an identified ligand, the existence of a second ligand that had not yet been identified or had not yet been matched with the GPCR, the GPCR still can be considered an orphan GPCR. Orphan GPCRs also include those GPCRs with no known sequence homology to another known GPCR.

As used herein, the term “functional conformation” refers to the ability of a receptor, such as a GPCR, to bind to a ligand.

As used herein, the term “substantially free from association with a lipid layer,” refers to the majority of receptors in a preparation being unassociated with a lipid layer.

There are several advantages to the use of a solubilized, immobilized, functional receptor according to methods of the invention. For example, substantially all contaminants and/or extraneous material, including non-target GPCRs, that typically interfere with conventional GPCR assays by creating a high level of “GPCR background” are absent from methods of the invention. GPCR background is found, for instance, in other systems such as those utilizing mammalian cells (intact or fragments therof). Assays in the art for isolating ligands that bind to a GPCR and for testing the function of a GPCR, such as, whole cell assays, are prone to a significant GPCR background problem because of the presence of unwanted GPCRs in the test system. That is, the test systems contain many endogenous GPCRs that complicate detecting the binding to or function of the GPCR of interest. For example, there is a significant background problem with HEK-293 cells, which are commonly used and which are known to have at least 15 GPCRs from multiple classes, many families and many more subfamilies. As such, methods of the invention allow for assays which detect the binding capabilities of or functions of the GPCR of interest without interference from other GPCRs. Also, utilizing functional, solubilized and immobilized GPCR to bind potential ligands from a complex mixture by affinity purification provides for more rapid and efficient assays than those currently available because the number of samples that are screened are reduced, and, as a result, extensive high-throughput screening is not typically necessary.

Additionally, natural ligands often are present in samples in low quantities (e.g., in the attomolar range) and, thus, can be difficult to detect and isolate. Accordingly, one approach described herein uses in vitro translation to overcome the challenge of identifying and isolating natural ligands for GPCRs (“de-orphaning”) when the ligands are present in a relatively small amount. Combining methods for immobilization of functional GPCRs with various approaches to the selection of members of translated cDNA pools (described further, below) which contain particular DNA sequences also can be of benefit in such situations in order to amplify a signal. Some of these cDNA selection approaches have been referred to as “sib selection.” This amplification arises (whether or not using cDNA pools) because even a single cDNA in a library that is translated can be detected if bound because the single cDNA can be amplified by making multiple copies of the cDNA prior to in vitro transcription and translation. As a result, although one copy of a gene is very difficult to detect from a cellular fraction, it can be amplified if using cDNA. Furthermore, the cDNA approach also makes identification of the bound ligand easier (for a proteinaceous ligand) because there is a direct link from the bound ligand to the sequence and, thus, the identity is determined in a more efficient manner. In other embodiments, however, methods of the invention further contemplate the use of protein sequencing using mass spectrometry.

Additionally, methods that involve sequencing cDNA to identify a ligand are simpler, faster, more reliable, and less expensive than protein sequencing using mass spectrometry.

In addition, a radiolabel can be incorporated into the in vitro translation step, highlighting the presence of a ligand and reducing the amount of ligand required to detect it.

Methods of the invention also involve the identification of GPCRs, such as orphan GPCRs, that can be expected to play a role in certain diseases and disorders. Compounds that are discovered to interact with orphan GPCRs can be useful as drugs in the diagnosis, prevention and treatment of diseases and disorders. For example, recently de-orphaned GPCRs include SLC-1 which is involved in obesity, and GPR14 which is involved in vasoconstriction (See e.g., Civelli O, et al., TINS 24(4):230-237 (2001). Also, for example, certain orphan GPCRs of interest that have not had their natural ligands identified are GPR38 and GPR39. See e.g., McKee K K, et al., Genomics 46(3):426-434. These orphan GPCRs are thought to be homologs of a growth hormone secretagogue receptor as determined by sequence homology.

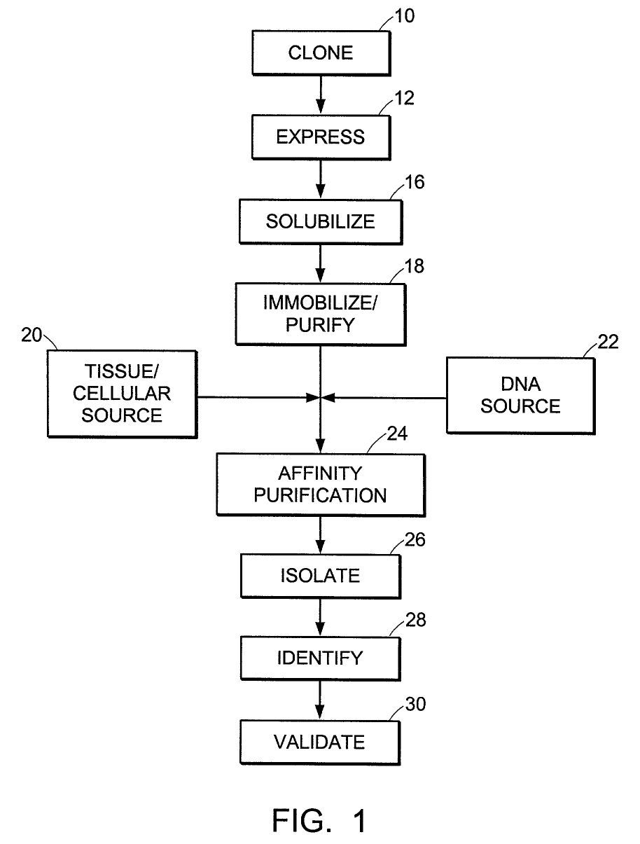

Generally, methods of the invention for identifying a ligand, such as a natural ligand, that has the ability to bind to a GPCR, such as an orphan GPCR, from a sample, such as a naturally-derived sample, include the steps outlined in FIG. 1. First, if not yet available, a GPCR is cloned 10 and expressed 12. During these steps, the GPCR can be “tagged” to facilitate its purification or isolation and/or the ligand's isolation. Next, the GPCR is solubilized 16, and purified and immobilized 18 on a support prior to mixing with potential ligands. As such, a GPCR in a functional conformation is associated with a support. Depending on certain criteria, including, but not limited to, the suspected characteristics of the natural ligand, the GPCR, the disease or disorder suspected to be involved with the GPCR, the cellular mechanism under consideration, and/or the characteristics of the tissue or cellular source or cDNA source, a naturally-derived sample is selected for testing. In one approach 20, the isolated GPCR can be mixed and interacted with the naturally-derived sample which can be a tissue or cellular source (for example, a cell culture medium). In another approach 22, the GPCR and can be mixed and interacted with a naturally-derived sample which can be a translated cDNA source. Next, affinity purification 24 is carried out such that the GPCR is bound to a natural ligand found in the selected naturally-derived source. Thereafter, ligands bound to target GPCR are isolated 26 (by separating the ligands from the support) and identified 28. Optionally, after the ligand is identified, the receptor is validated to confirm it as a potential disease target 30.

The general steps of a method according to the invention, as described in FIG. 1, are described in more detail, below. Additional detail of practicing the invention, including cloning, tagging, expressing, solubilizing, and immobilizing a GPCR can be found in the Examples below, and in U.S. Ser. No. 09/813,653, filed Mar. 20, 2001, U.S. Ser. No. 09/813,448, filed Mar. 20, 2001, and U.S. Ser. No. 09/813,651, filed Mar. 20, 2001. The disclosures of these three applications are incorporated by reference herein. In the first steps, a GPCR is cloned and/or expressed, depending upon whether such work has been done previously. To the extent cloning is necessary to obtain an expressed GPCR, the GPCR of interest can be isolated from a cDNA library. One way to accomplish cloning and expression is outlined below (and also in the Examples), but other methods are known to those skilled in the art. For example, isolation from the library is accomplished using oligo primers to the 5′ and 3′ ends of the gene of interest and PCR. The PCR product is adapted to homologous recombination vectors, such as, for example, Gateway vectors from Invitrogen (Carlsbad, Calif.) and Creator from Clontech (Palo Alto, Calif.), for expression by using an additional set of PCR oligo primers to add adaptor sequences that allow homologous recombination to occur with these vectors. A GPCR can be left un-tagged, or can be tagged by using tagging methods to generate a modified GPCR. A tagged GPCR functions to facilitate purification and isolation of the GPCR and/or isolation of the natural ligand. Generally, a tagged GPCR is a nucleic acid sequence corresponding to a GPCR fused to tag sequences (e.g., GST (glutathione transferase), FLAG, 6xHis, dual tagged with FLAG-GST, C-MYC, MBP (maltose binding protein), V5, Xpress, CBP (calmodulin binding protein), HA(hemagluttin)). Such fused GPCR sequences can include appropriate specific protease sites engineered into the vector. For example, one vector that can be used for homologous recombination with the PCR product is a vector incorporating a C-terminal GST tag and the necessary baculovirus promoters and other elements for expression in a baculovirus expression system in insect cells. This vector, with the GPCR cDNA inserted, is then homologously recombined with linear wild type baculovirus DNA to form a virus in cell culture. Accordingly, the virus infects insect cells, for example, whole Sf9 cells, High Five cells, or Sf21 cells, and thus produces more virus to amplify the virus and prepare the virus stock. The virus stock is used subsequently to infect the cells such as, for example, whole Sf9 cells, High Five cells, or Sf21 cells, in another infection round and prepare the protein of interest using the host cell machinery. Typically, expression takes 2 to 3 days.

Next, the GPCR is solubilized, purified and isolated. These steps can be accomplished by the following method, but other methods can be used. Additional information is found in the Examples, below. After incubating the virus with the cells for approximately 24 to 72 hours, the cells are harvested by centrifugation at approximately 800×g for approximately 10 minutes at approximately 4° C., washed with phosphate buffered saline, and flash-frozen in ethanol dry ice and stored at −80° C. until ready for use. The pellet is thawed on ice when before it is used. The pellet is processed by first resuspending the pellet in lysis buffer with homogenization. A typical lysis buffer is around neutral pH and contains a cocktail of protease inhibitors and detergent. For example, serine proteases, cysteine proteases, aspartyl proteases, and/or metalloproteases can be inhibited with inhibitors, such as, for example, PMSF, aprotinin, leupeptin, phenathroline, benzamidine HCl, and/or EDTA (ethylene diamine tetracetic acid). For example, detergents that can be used for the solubilization of the GPCR, include, but are not limited to, β-dodecylmaltoside, n-octyl-glucoside, CHAPS, deoxycholate, NP-40 (or Nonidet P-40, recently available under the trademark of Igepal CA-630 from Sigma, St. Louis, Mo.; chemical name (octylphenoxy)polyethoxyethanol, n˜9), Triton X-100, Tween-20, digitonin, Zwittergents, CYMAL, and lauroylsarcosine. Solubilization also can be conducted using varying NaCl concentrations to provide a GPCR in a functional conformation. Despite conventional thinking, the step of solubilization can be accomplished using low calcium or magnesium concentrations, low salt concentrations, and/or no salt concentrations, for example, low calcium and magnesium concentrations and no salt. Standard buffers such as PIPES (1,4-piperazine-diethanesulfonic acid) can be used. Also, HEPES (N-[2-Hydroxyethyl]piperazine-N′-[2-ethanesulfonic acid) can be used for solubilization with the detergent and no NaCl. In one embodiment, solubilization conditions are PIPES buffer, pH 7.5, 0.1%-0.3% NP-40, and protease inhibitor cocktail. Binding conditions can use the same buffer and detergent but with 3 mM CaCl₂ and 15 mM MgCl₂ added. Nuclear and cellular debris is removed by a low speed centrifugation. Membranes containing the receptor of interest are harvested from the remaining solution using high speed centrifugation.

After solubilization, a candidate for isolation is carried through for purification and isolation by immobilizing it to a support. Examples of such supports include, but are not limited to, any matrix, any resin, any bead or any column. Specific supports include, but are not limited to, glutathione-sepharose beads, sepharoses, agaroses including, for example, M2 FLAG, antibody resin, nickel columns, nitrocellulose or similar 2-dimensional matrices (including PVDF (polyvinylidene fluoride) membrane, and glass slides. This step can be accomplished as outlined below, but other methods can be used. After determining an appropriate detergent for solubilization and activity, such as, for example, NP-40, the GPCR is purified from the membrane fraction. Accordingly, the GPCR is free from association with a lipid layer. If a tag has been added to the expressed GPCR, the exact purification scheme typically depends on the tag construct chosen, which is subject to activity and ease of solubilization. For purification of the 6xHis-tagged receptor, the membrane fraction is loaded onto a Ni-NTA column (Qiagen, Valencia, Calif.) in the presence of detergent, such as, for example, NP-40, washed extensively, and eluted with imidazole. Purification of the FLAG-tagged receptor is performed using the anti-FLAG M2 affinity matrix (Sigma, St. Louis, Mo.) in the presence of detergent and eluted with glycine. Purification of a GST-tagged receptor is performed using a glutathione-sepharose column (Amersham Pharmacia Biotech, Piscataway, N.J.). The purification is performed in the presence of an appropriate detergent, such as, for example, NP-40, found for the system in the experiment described herein. The purification and isolation also can be conducted in the substantial absence of NaCl. Activity of the purified, immobilized receptors is assessed as described below.

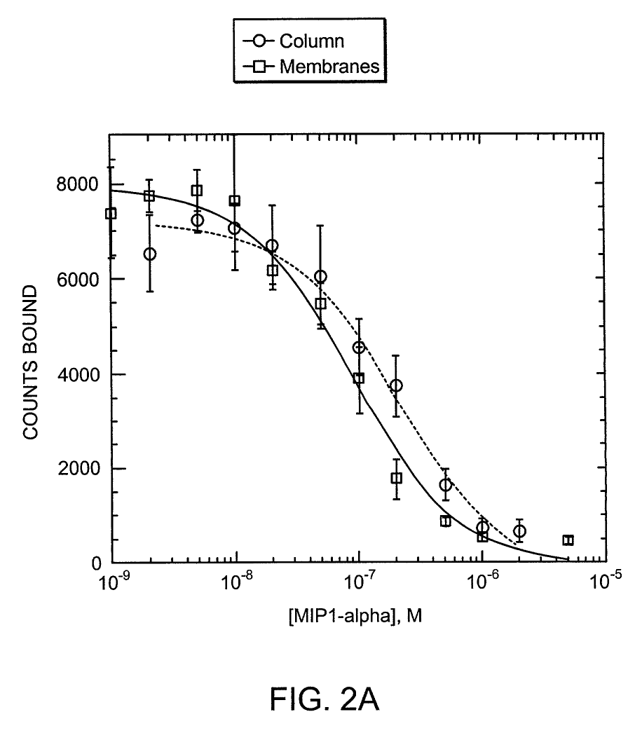

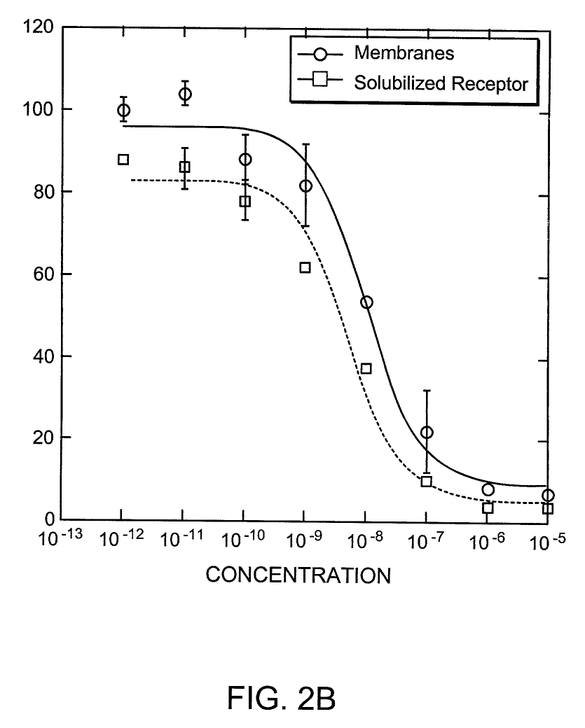

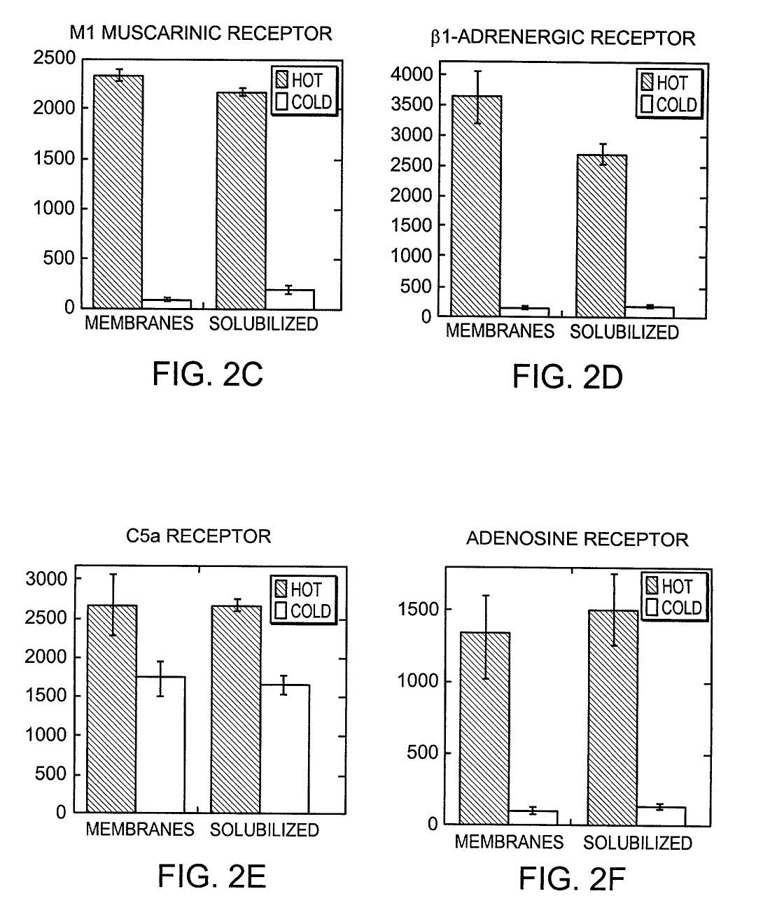

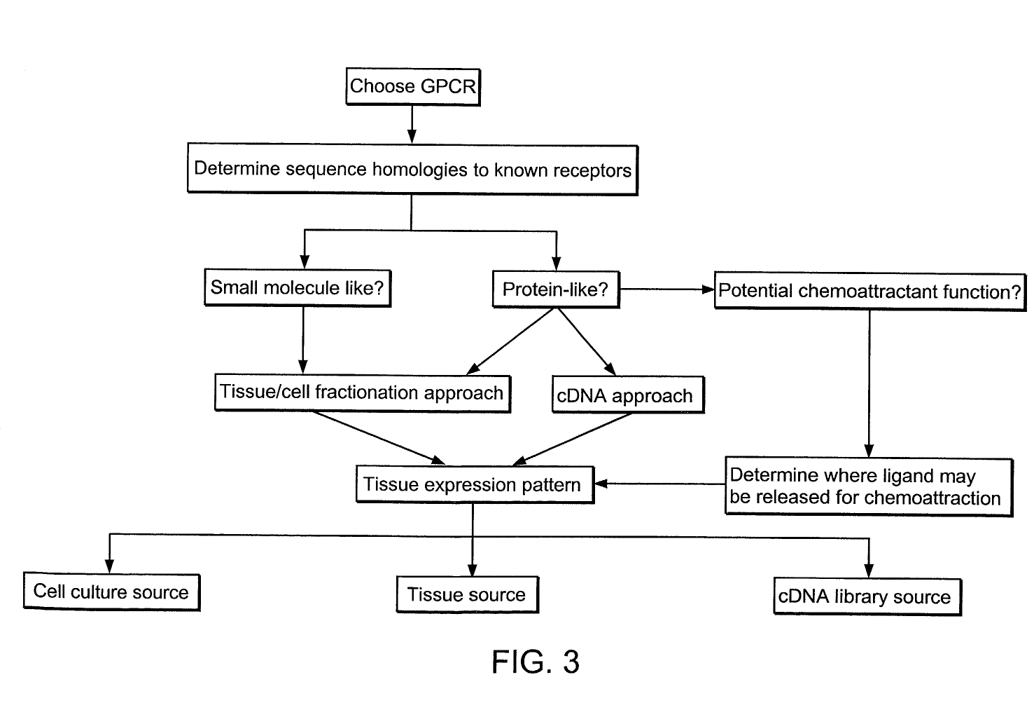

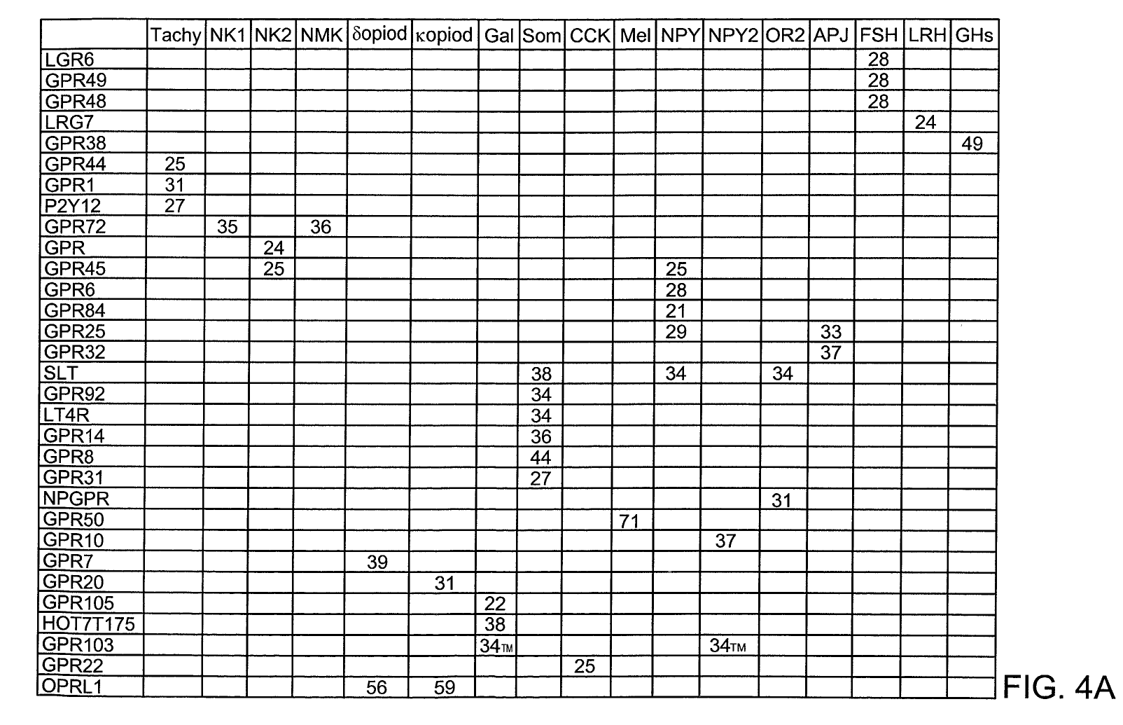

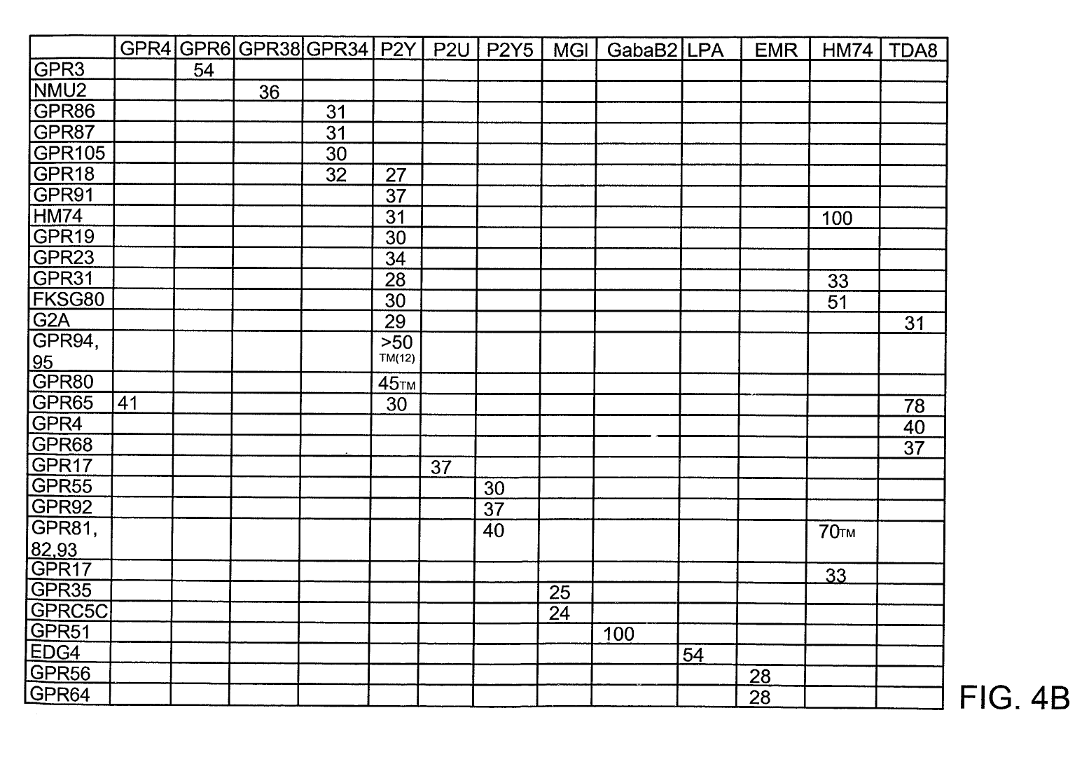

Solubilizing GPCRs, such as CCR5, CXCR4, muscarinic receptors, adenosine receptors, C5a receptor, and β-adrenergic receptor (which have different functions, belong to different classes, have different sequences, and have different structures), by utilizing a buffer with 0.0 mM NaCl in conjunction with NP-40, for example, provides such GPCRs in a functional conformation and provides such GPCRs in relatively high quantities with good activity. FIGS. 2A-2F provide a comparison of ligand binding to solubilized receptors in a functional conformation that are produced utilizing this method versus receptors still in membrane. These figures demonstrate that methods according to the invention produce GPCRs with a functional conformation.

For example, FIG. 2A shows binding-displacement curves for CCR5 (a GPCR) comparing the activity in the membranes (solid line, squares) with the activity of the CCR5 solubilized from the membranes and immobilized onto a resin in a column (glutathione sepharose; circles, dashed line). The CCR5 receptor, either in membranes or solubilized and immobilized onto the column, was incubated with the radiolabeled ligand, MIP-1β, at a set concentration, in the presence of various concentrations of the unlabeled ligand, MIP-1α. As the concentration of unlabeled ligand is increased (x-axis), it displaced more of the radiolabeled ligand, giving rise to fewer radioactive counts being bound to the receptor (y-axis). This displacement of the radioligand with increasing concentrations of unlabeled ligand gave a standard sigmoidal shaped curve. The two curves, one for the membrane binding-displacement and one for the binding-displacement for the immobilized receptor were substantially similar (within the experimental error), indicating similar functional conformation of the receptor in the membranes and immobilized receptor.

These general conditions that were used for solubilization have been applied to multiple and diverse GPCRs. Generally, the method was greater than about 80% successful in solubilizing and immobilizing GPCRs. FIGS. 2B-2F compare binding activity of several other GPCRs when such GPCRs are solubilized and immobilized as described above with the same GPCRs in a membrane and demonstrate that the binding activity of the solubilized receptors produced according to methods of the invention was substantially the same as the respective receptors tested while in a membrane. Accordingly, these GPCRs also are in a functional conformation. These experiments demonstrates the wide applicability of the solubilization conditions to provide GPCRs in a functional conformation.

More specifically, FIG. 2B shows a binding-displacement curve for the adenosine A1 receptor comparing the activity in the membranes (solid line, circles) with the activity of the solubilized adenosine A1 receptor (dashed line, squares). The adenosine A1 receptor, either in the membranes or solubilized, was incubated with the radiolabeled ligand, cyclopentyl-1,3-dipylxanthine, at a set concentration, in the presence of various concentrations of the unlabeled ligand (cyclopentyl-1,3-dipylxanthine). As the concentration of unlabeled ligand is increased (x-axis), it displaced more of the radiolabeled ligand, giving rise to fewer radioactive counts being bound to the receptor (y-axis). This displacement of the radioligand with increasing concentrations of unlabeled ligand gave a standard sigmoidal shaped curve. The two curves, one for the membrane binding-displacement and one for the binding-displacement for the solubilized receptor were substantially similar (within the experimental error), indicating that the solubilized receptor was in a functional conformation.

FIG. 2C shows the characterization of the M1 muscarinic receptor. FIG. 2C shows a binding-displacement histogram for the M1 muscarinic receptor comparing the activity in the membranes (left) with the activity of the solubilized M1 muscarinic receptor (right). The M1 muscarinic receptor, either in the membranes or solubilized (according to the invention), was incubated with the radiolabeled ligand, quinuclidinyl benzylate, at a set concentration, in the presence or absence of 10 μM unlabeled ligand (quinuclidinyl benzylate). As is the case with FIGS. 2D-2F, for FIG. 2C, the chart having columns designated with “Hot” refers to labeled (e.g. radiolabel) ligands, and the columns referred to as “Cold” refers to unlabeled ligands In the absence of unlabeled ligand, maximal binding of the radiolabeled ligand is observed. In the presence of unlabeled ligand, displacement of the radiolabeled ligand down to background levels is observed, demonstrating specificity of the binding activity. The binding and displacement of the ligand for the membranes and solubilized receptor are substantially similar (within the experimental error), indicating that the solubilized receptor was in a functional conformation.

FIG. 2D shows the characterization of the β1-adrenergic receptor. FIG. 2D shows a binding-displacement histogram for the β1-adrenergic receptor comparing the activity in the membranes (left) with the activity of the solubilized β1-adrenergic receptor (right). The β1-adrenergic receptor, either in the membranes or solubilized (according to the invention), was incubated with the radiolabeled ligand, iodocyanopindolol, at a set concentration, in the presence or absence of 10 μM unlabeled ligand (pindolol). In the absence of unlabeled ligand, maximal binding of the radiolabeled ligand is observed. In the presence of unlabeled ligand, displacement of the radiolabeled ligand down to background levels is observed, demonstrating specificity of the binding activity. The binding and displacement of the ligand for the membranes and solubilized receptor are substantially similar (within the experimental error), indicating that the solubilized receptor was in a functional conformation.

FIG. 2E shows the characterization of the C5a receptor. FIG. 2E shows a binding-displacement histogram for the C5a receptor comparing the activity in the membranes (left) with the activity of the solubilized C5a receptor (right). The C5a, either in the membranes or solubilized (according to the invention), was incubated with the radiolabeled ligand, C5a, at a set concentration, in the presence or absence of 10 μM unlabeled ligand (C5a). In the absence of unlabeled ligand, maximal binding of the radiolabeled ligand is observed. In the presence of unlabeled ligand, displacement of the radiolabeled ligand down to background levels is observed, demonstrating specificity of the binding activity. The binding and displacement of the ligand for the membranes and solubilized receptor are substantially similar (within the experimental error), indicating that the solubilized receptor was in a functional conformation.

FIG. 2F shows the characterization of the adenosine A1 receptor. FIG. 2F shows a binding-displacement histogram for the adenosine A1 receptor comparing the activity in the membranes (left) with the activity of the solubilized adenosine A1 receptor (right). The adenosine A1 receptor, either in the membranes or solubilized (according to the invention), was incubated with the radiolabeled ligand, cyclopentyl-1,3-dipylxanthine, at a set concentration, in the presence or absence of 10 μM unlabeled ligand (cyclopentyl-1,3-dipylxanthine). In the absence of unlabeled ligand, maximal binding of the radiolabeled ligand is observed. In the presence of unlabeled ligand, displacement of the radiolabeled ligand down to background levels is observed, demonstrating specificity of the binding activity. The binding and displacement of the ligand for the membranes and solubilized receptor are substantially similar (within the experimental error), indicating that the solubilized receptor was in a functional conformation.

Based upon the experimental results shown in FIGS. 2A-2F, solubilization conditions were found to be widely applicable. These conditions were tested using a variety of GPCRs from a variety of classes with different applications, having a variety of structures, and having a variety of ligands (small molecule amines, nucleotides, peptides, and proteins). Testing such a wide variety of GPCRs spanning the range of certain properties GPCRs can have indicates the likelihood that these conditions will be applicable to most, if not all, GPCRs. Accordingly, methods of the invention can produce GPCRs in a functional conformation.

Next a naturally-derived sample containing potential ligands (e.g., natural ligands) is bound to the GPCR. Such a sample can be obtained from naturally-derived sources which include, but are not limited to an inactive, precipitated protein preparation, a nucleic acid (such as a cDNA), a translated nucleic acid (such as a cDNA), a cDNA library encoding a set of at least two proteins, a cell, a whole cell preparation, a cell membrane preparation, a cell culture medium, a cell line, a tissue, a tissue extract and fractions thereof, an extract of cells grown in tissue culture and fractions thereof, and an organism. Two general categories of these naturally-derived sources include (1) tissue or cellular sources and (2) cDNA sources.

Alternatively, one can choose certain naturally-derived samples for screening. In order to choose a naturally-derived sample to screen for a GPCR ligand, certain parameters can be considered. The skilled artisan will appreciate that all that is required is routine experimentation and routine skill to select a naturally-derived sample. Any naturally-derived sample, or multiple naturally-derived samples, can be screened in a “shot-gun” approach.

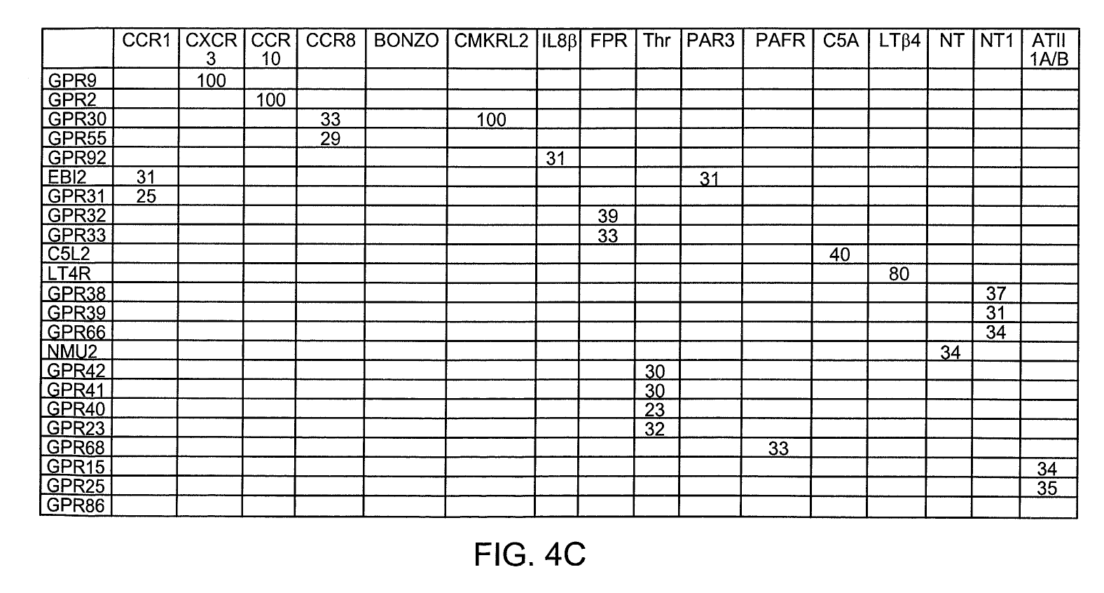

To the extent it is desired to select a particular naturally-derived sample, an exemplary schematic diagram is provided, for example, in FIG. 3, which outlines certain considerations involved in the selection of a naturally-derived sample. After choosing the GPCR of interest, one consideration is to compare the sequence homology or identity between a GPCR of interest (for example, an orphan GPCR) and the sequence of a known receptor. At least about 10%, more preferably about 20%, more preferably about 30%, more preferably about 40%, more preferably about 50%, more preferably about 60% or more identity between the two sequences can suggest choosing a naturally-derived sample from the tissue or cellular source or cDNA library thought or known to contain the known GPCR or its ligand. Sequence homology (rather than strict identity) also can be used. At least about 50%, more preferably about 60%, more preferably about 70%, more preferably about 80%, and more preferably about 90% or more sequence homology can suggest a choice of a naturally-derived sample. Below, sequence identity is discussed, but a similar analysis applies to sequence homology.

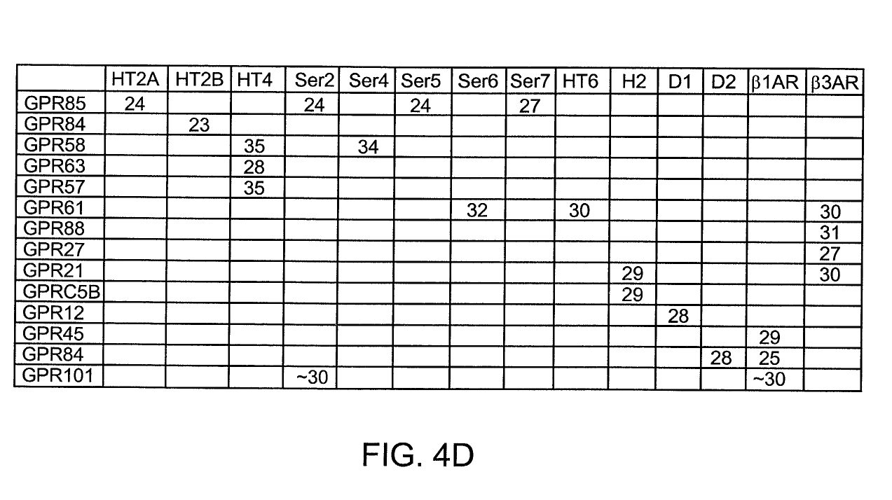

More specifically, the sequence identities between the orphan GPCR and known receptors are compared. This information can suggest that an orphan receptor is a member of a specific class or is a member of an entirely new class. An example of certain sequence identity comparisons of orphan GPCRs to other GPCRs are provided, for example, in tables in FIGS. 4A-4D. One skilled in the art can construct similar tables for additional GPCRs without undue experimentation. The tables in FIGS. 4A-4D include various orphan and known GPCRs listed as the headings of each of the columns and include orphan GPCRs listed as the headings for each row. Table 1, below, provides a key, which lists the fill names of the GPCRs which are abbreviated in FIGS. 4A-4D. The tables in FIGS. 4A-4D show percent sequence identity for each orphan GPCR compared to other known or orphan GPCRs to help classify them as a family. Percent sequence identity is the number of amino acids which are determined to be identical in identical positions in the two receptors after alignment. The table correlates the percent sequence identify between any orphan GPCR and any other GPCR with any significant homology (greater than about 20% identity in this instance). For example, as seen by aligning GPR38 with GHS in the table in FIG. 4A, 49% of the amino acids are identical to each other in terms of position and identity. Also, the TM subscript as used in the table in FIGS. 4A-4D is used to designate that the percent identity reported is only in the transmembrane regions and that the loop regions are not compared. Also, if the class of the known GPCR is known, or the class of other GPCRs in which the known GPCR resides, is known, then, if the orphan GPCR has some identity to the known GPCR, it can be inferred that useful naturally-derived samples can be chosen from those limited number of tissue or cellular sources or cDNA libraries relevant to a particular class of GPCR. Also, the following orphan receptors have no known homology to any identified GPCRs as provided in the table in FIGS. 4A-4D: RDC1, GPR37, CMLKR1, GPR26, GPR43, GPR75, GPR34, GPR78, GPR84, GPR85, and GPR90.

TABLE 1
Key for Figures 4A-4D

| Abbreviation | Full Name |
|---|---|
| Tachy | Tachykinin receptor (drosophila) |
| NK1, NK2 | Neurokinin receptors 1 & 2 |
| NMK | Neuromedin K receptor |
| Gal | Galanin receptor |
| Som | Somatostatin receptor |
| CCK | Cholecystokinin receptor |
| Mel | Melanocortin receptor |
| NPY | Neuropeptide Y receptor |
| OR2 | Orexin 2 receptor |
| FSH | Follicle stimulating hormone receptor |
| LRH | Lutropin-choriogonadotropin hormone receptor |
| GHs | Growth hormone secetagogue receptor |
| P2Y, P2U, P2Y5 | Purinergic receptors |
| MG1 | Metabotropic glutamate receptors |
| LPA | Lysophosphatidic acid receptor (also EDG) |
| FPR | Formylpeptide receptor |
| Thr | Thrombin receptor |
| PAR | Protease activated receptor |
| PAF | Platelet activating factor |
| LTβ4 | Leukotriene β4 receptor |
| NT | Neurotensin receptor |
| ATII 1A/B | Angiotensin type II receptor 1A or 1B |
| HT | Hydroxytryptamine receptor |
| Ser | Serotonin receptors |
| H2 | Histamine 2 receptor |
| D1, D2 | Dopamine receptors 1 & 2 |
| β1AR, β3AR | β-adrenergic receptors 1 & 3 |

To determine whether an orphan GPCR (“candidate”) has the requisite percentage similarity or identity to a known reference GPCR (“reference”) (or an orphan reference GPCR), the candidate amino acid sequence (or a portion thereof) and the reference amino acid sequence (or a portion thereof) are first aligned using the dynamic programming algorithm described in Smith and Waterman (1981), J. Mol. Biol. 147:195-197, in combination with the BLOSUM62 substitution matrix described in FIG. 2 of Henikoff and Henikoff (1992), “Amino acid substitution matrices from protein blocks”, PNAS (November 1992 ), 89:10915-10919. For the present invention, an appropriate value for the gap insertion penalty is −12, and an appropriate value for the gap extension penalty is −4. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and reference sequence is made, a percent similarity score can be calculated. The individual amino acids of each sequence are compared sequentially according to their similarity to each other. If the value in the BLOSUM62 matrix corresponding to the two aligned amino acids is zero or a negative number, the pairwise similarity score is zero; otherwise the pairwise similarity score is 1.0. The raw similarity score is the sum of the pairwise similarity scores of the aligned amino acids. The raw score is then normalized by dividing it by the number of amino acids in the smaller of the candidate or reference sequences. The normalized raw score is the percent similarity. Alternatively, to calculate a percent identity, the aligned amino acids of each sequence are again compared sequentially. If the amino acids are non-identical, the pairwise identity score is zero; otherwise the pairwise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity. Accordingly, gap penalties are not used in this calculation, although they are used in the initial alignment.

Additionally, if there is some sequence identity between the orphan GPCR and other known (or orphan) receptors, and if the class of the known GPCR (or other orphan GPCR) is known, then it is likely that ligands within the class are similar. As such, it can be inferred that a ligand in a naturally-derived sample can be obtained from a limited number of tissue or cellular sources or cDNA libraries relevant to the particular class. Also, the similarity of ligands in a particular class is useful to limit the number of naturally-derived samples from which the ligand can be obtained. For example, one considers whether the known GPCR (or its class) to which the orphan GPCR has some identity has ligands that are small molecule-like, or are protein-like, whether the known GPCR (or its class) has any potential chemoattractant functions, and whether the known GPCR (or its class) or an orphan GPCR is likely to bind, for example, to a peptide, protein, nucleotide, small biogenic amine or molecule. For orphan GPCRs with potential peptide or protein ligands, methods utilizing cDNA libraries or tissue or cellular sources can be used. However, for orphan GPCRs thought to have a ligand which is a small molecule or is otherwise a non-proteinaceous molecule, the cDNA approach is not used and, instead, tissue or cellular sources are used.

For an orphan GPCR that has some identity to a chemokine or chemoattractant receptor (or similar receptor), another approach can be used. These receptors are found on one cell and respond to ligands from a distant site causing the migration of the cell (or a cell part) to that distant site. These responses can be both positive (e.g., to fight infection or inflammation) and negative (e.g., the metastasis of tumor cells). For receptors such as these, one can use information from known systems to identify the distant site. For example, if there is some identity between an orphan receptor and a known receptor (or a receptor in its class), then one can study the systems of which the known receptor (or a receptor in its class) is a part to determine a limited number or relevant tissue or cellular sources or cDNA libraries that are relevant. In addition, since the ligands of these receptors are generally proteinaceous in nature, the cDNA library has significant advantages over other tissue and cellular naturally-derived samples, allowing many more potential ligands to be screened.

Another consideration is the tissue expression pattern of the orphan GPCR. The tissue in which the orphan GPCR is most highly expressed can be identified using, for example, PCR, western blots, and/or in situ hybridizations. Determining the tissue expression pattern of the receptor can limit naturally-derived samples, for example a tissue or cellular source or a cDNA library, which are useful for screening by only screening those in which the orphan GPCR is expressed. For example, if a receptor is only expressed in a certain region of the brain, it is likely that this region of the brain is one tissue to use as a ligand source. After the tissue in which the receptor of interest is expressed is identified, the tissue can be used to identify the natural ligand, or the tissue expression profile leads to rational determination of the tissue or cells to use.

Another consideration is to determine whether the tissue or cellular source or cDNA source is readily available and/or is a good candidate for experimentation. For example, certain cells or tissues grow better than others and/or are easier to manipulate, such as, for example, when inducing or adding growth factors. Monocytic cells, for example, are easily manipulated with lipopolysaccharide (LPS) to stimulate massive infection, which produces a heightened chemokine response.

Once, a naturally-derived sample is chosen, the GPCR of interest (for example, an orphan GPCR) can be interacted with the naturally-derived sample to bind a molecule (e.g., a natural ligand) in the sample to the GPCR. The GPCR of interest is immobilized on (or otherwise associated with) a support for use in capturing the natural ligand. Examples of such supports include, but are not limited to, any matrix, any resin, any bead, or any column. Specific supports include, but are not limited to, glutathione-sepharose beads, sepharoses, agaroses including, for example, M2 FLAG, antibody resin, nickel columns, nitrocellulose or similar 2-dimensional matrices (including PVDF (polyvinylidene fluoride) membrane, and glass slides. Once immobilized and in a functional conformation GPCRs can be mixed with, for example, tissue extracts, fractions from tissue extracts, cell culture media, extracts from cells grown in tissue culture, fractions from extracts from cells grown in tissue culture, and proteins translated from small pools of cDNA from cDNA libraries, for the affinity purification. This step can be accomplished by the following method, but other methods can be used. Additional detail can be found in the Examples, below.

If a tissue or cellular source is chosen as a sample, tissue or cell extracts can be prepared by homogenization of the tissue or cells in biological buffers such as phosphate buffered saline. Soluble extracts can be separated using centrifugation. The insoluble material can be further processed using salt washes to release membrane-associated proteins or ligands. Prior to this, if the ligand of interest may be found in other cellular locations such as the nucleus or mitochondria, these fractions can be prepared also by differential centrifugation. In addition, membrane reagents can be detergent solubilized, or organic hydrophobic solvents can be used for small hydrophobic molecules. Each of these extracts can be applied separately to the immobilized, functional receptor and any specifically-bound compounds can be isolated by separating such compounds from the support. Each of these extracts can be further broken down into smaller subsets using, for example, high performance liquid chromatography (“HPLC”) and collecting fractions. Thereafter, the fractions of these extracts can be incubated with the immobilized, functional receptor, and the specifically-bound compounds can be identified. In all cases, the fractionation steps are kept as simple as possible to minimize the loss or alteration of a potential ligand.

When a tissue or cellular source (such as tissue extracts or fractions thereof) is applied to an immobilized GPCR on a support in a functional conformation to allow the two to interact, bound molecules are captured and are separated from the support for further isolation and identification. For example, after washing, bound ligands can be eluted from the column by boiling in SDS-PAGE sample buffer or by other means such as high salt, low pH, and/or specific methods that would also elute the bound tagged receptor (such as glutathione for a GST-tagged receptor). Next, the isolated ligands can be submitted individually to mass spectrometry and/or to sequencing by Edman degradation for characterization.

More particularly, in one example, SDS-PAGE sample buffer is added to the receptor-ligand-support complex directly; the sample buffer is boiled to release the ligand from the support (whether or not the ligand is released from the receptor); and the boiled sample buffer is run out on a SDS-PAGE gel. Proteins on the gel can be silver stained (when the ligand is proteinaceous), cut out from the gel, and submitted for sequencing by mass spectrometry. The addition of SDS-PAGE followed by boiling the sample buffer containing the receptor-ligand-support complex separates the molecule from the support. The sequence obtained can be used to search the protein (and translated genome) databases for a match for identification. If a ligand is a small molecule, non-proteinaceous in nature, the ligand-receptor-support complex can be loaded onto an HPLC column for separation from the support and isolation. This sample can be directly analyzed by a mass spectrometer (HPLC-MS), and the ligand can be identified by fragmentation using mass spectrometry.

If a cDNA library is chosen as a sample, in vitro transcription and translation is performed using a transcription/translation system, such as, with rabbit reticulocytes or with a wheat germ system (for example, TNT Coupled Wheat Germ Extract System from Promega, Madison, Wis.). The wheat germ system couples transcription and translation together to reduce the time required for the procedure. The cDNA template (for example, a pool of about 100 cDNAs from a cDNA library, although more or less than 100 cDNAs can be used) is added to the wheat germ extract, along with buffer, polymerase, amino acid mixture, radiolabeled amino acid (typically [³⁵S-Met], RNase inhibitor, and nuclease-free water. The reaction mixture is incubated at 30° C. for 2 hours. The in vitro translation mixture then is incubated with the immobilized GPCR in a functional conformation for approximately 30 minutes to allow the two to interact, followed by washing with buffer to remove non-specific binding. SDS-PAGE sample buffer is then added directly to the immobilized receptor and bound ligand, boiled for 1 minute to separate the bound ligand from the support, and loaded directly onto the SDS-PAGE gel. These materials are run out on a SDS-PAGE gel, the gel is dried, and the radiolabeled products are detected by phosphorimage. If a pooling procedure is used, positive pools of cDNA clones can be replated at, for example, about 10 cDNA clones per preparation, and the procedure is repeated. Any preparation that is positive is then replated again with fewer cDNA clones per sample. This procedure is continued until a single cDNA clone that encodes a ligand that binds to the GPCR is identified.

A method similar to this can be used for identification of transcriptional activators and genes involved in cell cycle control, apoptosis, and early development. For an orphan GPCR, methods according to the invention allow for screening many proteins expressed in various tissues at different levels to identify rapidly novel proteins which bind the receptor with high affinity. By using immobilized receptors, the process is efficiently performed with low or no background from non-target GPCRs. Also, by including a radiolabel or fluorescent label in the in vitro translation reaction, a simple and sensitive method of detection is built-in for the binding of the protein to the receptor.

Alternatively, after immobilization onto the affinity resin and after the natural ligand is bound to the receptor, a ligand can be separated from the support by cleaving the tag on the GPCR using a specific protease (as designed into the protein/vector) to release the complex. Also, in certain methods of the invention, the ligand is left bound to the receptor. Any non-specific binders are washed off with buffer or high affinity binders are selected for by using increasing concentrations of salt in the washes. Following several washes, the bead/resin-receptor-bound ligand complex is loaded directly onto an SDS-PAGE (sodium dodecyl sulfate polyacrylamide gel electrophoresis ) gel by adding a typical sample buffer, boiling 1 minute (separating the molecule from the support), centrifuging out the beads/resin, and loading all of the sample (receptor and any bound ligand). The sample buffer contains a neutral tris buffer, bromophenol blue as a dye used to follow the migration, glycerol or sucrose to sink the sample into the wells such that it doesn't mix with the other samples, and SDS to solubilize and give a negative charge to all the proteins such that they run according to size only, not charge. This method allows the least amount of sample manipulation minimizing the chance of loss of any potential ligands, although elution can be used in circumstances where necessary or other advantages require it.

To enhance the specificity of the ligand obtained from the affinity purification from a sample, other methods can be used. The bound components of the fractions or extracts can be eluted from the immobilized protein with specific N-terminally blocked peptides or other non-sequenceable analogs. In order to ensure that non-specific binding of ligands does not occur with the support, the affinity tagged protein can be eluted with its bound ligand. This elution elutes only the tagged receptor and its biding partner. For example, to avoid the release of minor contaminants from the affinity resin after binding of the ligand, the release/elution of the tagged GPCR with its bound ligand is accomplished using specific N-terminally blocked peptides or other non-sequenceable analogs. This is accomplished using acetylated FLAG peptide to elute GPCR-FLAG receptor from the resin. Alternatively, the tag from GPCR is cleaved using a specific protease (as designed into the protein/vector, for example, either enterokinase or thrombin) after immobilization onto the affinity resin and after the ligand is bound to release the complex.

Optionally, the next step can be the validation or confirmation of that target the GPCR has a role in a particular disease or disorder. Once the ligand is identified, the pathway in which the target GPCR is involved can be confirmed or, optionally, identified if unknown. Typically, with orphan GPCRs, the receptor's role in a disease or disorder is unidentified. Therefore, a further inquiry after the ligand is identified is made to confirm or validate the target GPCR's role or function in a particular disease or disorder pathway. A mouse or other animal model, including a human model, can be used to perform validation studies.

For example, transgenic knockouts can be prepared as soon as the receptor is identified from the genome, however it is difficult to know any potential secondary affects from this without knowing the ligand and pathway potentiated by binding of the ligand. However, identification of an inhibitor can enable specific validation of a target in a disease model. Inhibitors are desired because they can block the activity of a GPCR (similar to a knockout) but they have many fewer potential secondary effects. As only the specific interactions are prevented, any secondary interactions such as scaffolding functions remain (unlike transgenic knockouts). Once an inhibitor is identified, the inhibitor can be directed to an in vivo model system, such as a specifically developed mouse model, to look at the direct effects of not having the specific function indicated by the specific inhibitor. Accordingly, the isolated natural ligand can be used to set up various screens for an inhibitor of the receptor. These screens can include assays known to those skilled in the art, such as binding assays or functional assays. One assay uses the receptor with the ligand, radiolabeled, along with libraries of inhibitors to look for inhibition of binding of the natural ligand. Another assay that can be used is a signaling assay in which the natural ligand is used to create a signal such as calcium mobilization (detected by a standard fluorescent assay), and inhibitors are screened for by their ability to inhibit or diminish the calcium signal.

Also, to validate a GPCR as a target for drug discovery for a particular disease or disorder, there are at least two possibilities: the use of the natural ligand for treatment (as described above) or the use of an inhibitor for the treatment of a disease or disorder. If treatment is possible using the natural ligand, the target validation is performed directly with the natural ligand. If the envisioned treatment involves the use of an inhibitor, then the target validation is performed using an inhibitor. In this case, the inhibitor must be synthesized and characterized, for example by using a standard binding inhibition assay. Once the inhibitor or natural ligand (depending on the therapeutic approach) is confirmed to be potent and specific, the target validation is begun by performing a cellular assay using cells from a particular disease model with the addition of natural ligand or inhibitor to validate the specific role of the receptor of interest by looking for prevention of a transformation of the cell to the specific disease state or phenotype. After validating or confirming the target GPCR using assays, target validation can be performed using animal models. A disease animal model is chosen for the therapeutic target of interest and either the natural ligand or the inhibitor is provided (typically by injection). The prevention or treatment of the disease in the model is assessed by the phenotype after treatment compared to untreated controls. If the treatment is effective, then the target is valid for the particular disease. The ligand or inhibitor can then be a drug candidate as well, depending on its pharmacological properties (such as, for example, toxicity, availability, half-life, and potency).

After the identification of a ligand of a GPCR of interest, and optionally, the validation or confirmation of the ligand as a potential binding therapeutic, natural ligands and related molecules can serve as a lead compound and/or be formulated as a drug. In general, binding therapeutics are useful in the prevention and treatment of diseases and disorders. The diseases and disorders (with exemplary potential targets in parentheses), include, heart disease (angiotensin II receptor, β-adrenergic receptors); asthma (leukotriene receptors, CysLT1R); rheumatoid arthritis (CCR5, BLT1); multiple sclerosis (CCR5. CCR2); obesity (MCHR, melatonin receptor, neuropeptide Y receptors 1 and 2); reproduction disorders (LRH, FSH); gastrointestinal disorders (cholecystokinin receptor, somatostatin receptor); depression (serotonin receptors, neuropeptide Y receptors 1 and 2); hypertension (orexin receptor, APJ receptor); infectious diseases (CCR5, CXCR4); thrombosis (thrombin receptor, PAR3); inflammation (leukotriene receptors, PAFR); cancer (CXCR4, CCR7, neurotensin receptors); stroke (metabotropic glutamate receptors); neurological disorders (dopamine receptors, serotonin receptors); ulcers (H2 receptors); Parkinson's disease (dopamine receptors); and, pain treatment (opioid receptors, neurokinin receptors). Other diseases and disorders as well as examples of their respective potential targets are provided in the following Table 2. This table is exemplary and not meant to be an exhaustive list. For example, multiple sclerosis can be treated by combining isolated ligands or molecules identified with other compounds, for example, combining identified GPCR inhibitors and interferons. Also, the newly identified natural ligand can also be used directly as a therapeutic treatment for a disease of disorder. Examples of this include the treatment of reproductive disorders using follicle stimulating hormone, the treatment of Parkinson's disease with dopamine, and the treatment of depression with serotonin.

TABLE 2
Disease and Disorders Matched with GPCRs

| Disease/Disorder | GPCRs |
|---|---|
| Heart disease | ATII |
| | β-AR |
| Asthma | Leukotriene receptors |
| | CysLT1 |
| Hypertension | Orexin receptors |
| | APJ receptor |
| | Bradykinin receptor |
| Stroke | Metabotropic glutamate |
| | receptors |
| Rheumatoid arthritis | CCR5 |
| Multiple sclerosis | CCR5 |
| Obesity | MCHR |
| | Melatonin receptors |
| | Neuropeptide Y receptors |
| Reproduction disorders | LRH |
| | FSH |
| Gastrointestinal disorders | CCKR |
| | Somatostatin receptors |
| | Bombesin receptor |
| Ulcers | H2 receptor |
| Depression | Serotonin receptors |
| | Neuropeptide Y receptors |
| Infectious disease | CCR5 |
| | CXCR4 |
| Thrombosis | Thrombin receptor |
| | PAR3 |
| Inflammation | Leukotriene receptors |
| | PAFR |
| Cancer | CXCR4 |
| | CCR7 |
| | Bradykinin receptor |
| Pain | Opioid receptors |
| | Neurokinin receptors |
| | Bradykinin receptor |
| Parkinson's | Dopamine receptors |
| Neurotransmission | mAchR |

The identification of candidates that, alone or admixed with other suitable molecules, are competent to inhibit GPCR binding are contemplated by the invention. Further, the production of commercially significant quantities of the aforementioned identified candidates, which are suitable for the prevention and/or treatment of certain diseases and disorders is contemplated. Moreover, the invention provides for the production of therapeutic grade commercially significant quantities of GPCR binding antagonists, agonists or derivatives in which any undesirable properties of the initially identified analog, such as in vivo toxicity or a tendency to degrade upon storage, are mitigated.

Methods of preventing and treating diseases and disorders also, after the identification of a peptide, peptidomimetic, or small molecule agonist or antagonist of GPCR binding activity, include the step of administering a composition including such a compound capable of inhibiting GPCR binding as described herein.

Nucleic acid molecules (including DNA, RNA, and nucleic acid analogs such as PNA) which are themselves active or which code for active expressed products; peptides; proteins; antibodies; or other chemical compounds isolated and identified, or based upon or derived from ligands isolated and identified according to the invention (also referred to as active compounds or drugs) can be incorporated into pharmaceutical compositions suitable for administration. Such active compounds or drugs include inhibitors identified or constructed as a result of isolating and identifying ligands according to the invention. The drug compounds discovered according to the present invention can be administered to a mammalian host by any route. Thus, as appropriate, administration can be oral or parenteral, including intravenous and intraperitoneal routes of administration. In addition, administration can be by periodic injections of a bolus of the drug, or can be made more continuous by intravenous or intraperitoneal administration from a reservoir which is external (e.g., an i.v. bag). In certain embodiments, the drugs of the instant invention can be therapeutic-grade. That is, certain embodiments comply with standards of purity and quality control required for administration to humans. Veterinary applications are also within the intended meaning as used herein.

The formulations, both for veterinary and for human medical use, of the drugs according to the present invention typically include such drugs in association with a pharmaceutically acceptable carrier therefor and optionally other therapeutic ingredient(s). The carrier(s) can be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified according to the invention and/or known in the art) also can be incorporated into the compositions. The formulations can conveniently be presented in dosage unit form and can be prepared by any of the methods well known in the art of pharmacy/microbiology. In general, some formulations are prepared by bringing the drug into association with a liquid carrier or a finely divided solid carrier or both, and then, if necessary, shaping the product into the desired formulation.

A pharmaceutical composition of the invention is formulated to be compatible with its intended route of administration. Examples of routes of administration include oral or parenteral, e.g., intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., 1990. Formulations for parenteral administration also can include glycocholate for buccal administration, methoxysalicylate for rectal administration, or cutric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions that are solid at room temperature and liquid at body temperatures. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration can be in the form of discrete units such as capsules, gelatin capsules, sachets, tablets, troches, or lozenges, each containing a predetermined amount of the drug; in the form of a powder or granules; in the form of a solution or a suspension in an aqueous liquid or non-aqueous liquid; or in the form of an oil-in-water emulsion or a water-in-oil emulsion. The drug can also be administered in the form of a bolus, electuary or paste. A tablet can be made by compressing or moulding the drug optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Moulded tablets can be made by moulding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition can be sterile and can be fluid to the extent that easy syringability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug which can be in microcrystalline form, for example, in the form of an aqueous microcrystalline suspension. Liposomal formulations or biodegradable polymer systems can also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pasts; or solutions or suspensions such as drops. Formulations for topical administration to the skin surface can be prepared by dispersing the drug with a dermatologically acceptable carrier such as a lotion, cream, ointment or soap. In some embodiments, useful are carriers capable of forming a film or layer over the skin to localize application and inhibit removal. Where adhesion to a tissue surface is desired the composition can include the drug dispersed in a fibrinogen-thrombin composition or other bioadhesive. The drug then can be painted, sprayed or otherwise applied to the desired tissue surface. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, such as for asthma, inhalation of powder (self-propelling or spray formulations) dispensed with a spray can, a nebulizer, or an atomizer can be used. Such formulations can be in the form of a finely comminuted powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from a pressured container or dispenser which contains a suitable propellant, e.g., a gas such as carbon dioxide, or a nebulizer. Nasal drops also can be used.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and filsidic acid derivatives. Transmucosal administration can be accomplished through the use of nasal sprays or suppositories. For transdermal administration, the active compounds typically are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Microsomes and microparticles also can be used.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, the drugs identified according to the invention can be formulated for parenteral or oral administration to humans or other mammals, for example, in therapeutically effective amounts, e.g., amounts which provide appropriate concentrations of the drug to target tissue for a time sufficient to induce the desired effect. Additionally, the drugs of the present invention can be administered alone or in combination with other molecules known to have a beneficial effect on the particular disease or indication of interest. By way of example only, useful cofactors include symptom-alleviating cofactors, including antiseptics, antibiotics, antiviral and antifungal agents and analgesics and anesthetics. Where a peptide, peptidomimetic, or small molecule agonist or antagonist of GPCR binding activity or drug therefrom identified according to the invention is to be used as part of a transplant procedure, it can be provided to the living tissue or organ to be transplanted prior to removal of tissue or organ from the donor. The drug can be provided to the donor host. Alternatively or, in addition, once removed from the donor, the organ or living tissue can be placed in a preservation solution containing the drug. In all cases, the drug can be administered directly to the desired tissue, as by injection to the tissue, or it can be provided systemically, either by oral or parenteral administration, using any of the methods and formulations described herein and/or known in the art.

Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution. Generally, an organ preservation solution usually possesses one or more of the following properties: (a) an osmotic pressure substantially equal to that of the inside of a mammalian cell (solutions typically are hyperosmolar and have K+ and/or Mg++ ions present in an amount sufficient to produce an osmotic pressure slightly higher than the inside of a mammalian cell); (b) the solution typically is capable of maintaining substantially normal ATP levels in the cells; and (c) the solution usually allows optimum maintenance of glucose metabolism in the cells. Organ preservation solutions also can contain anticoagulants, energy sources such as glucose, fructose and other sugars, metabolites, heavy metal chelators, glycerol and other materials of high viscosity to enhance survival at low temperatures, free oxygen radical inhibiting and/or scavenging agents and a pH indicator. A detailed description of preservation solutions and useful components can be found, for example, in U.S. Pat. No. 5,002,965, the disclosure of which is incorporated herein by reference.

The effective concentration of the drugs identified according to the invention that is to be delivered in a therapeutic composition will vary depending upon a number of factors, including the final desired dosage of the drug to be administered and the route of administration. The preferred dosage to be administered also is likely to depend on such variables as the type and extent of disease or indication to be treated, the overall health status of the particular patient, the relative biological efficacy of the drug delivered, the formulation of the drug, the presence and types of excipients in the formulation, and the route of administration. In some embodiments, the drugs of this invention can be provided to an individual using typical dose units deduced from the earlier-described mammalian studies using non-human primates and rodents. As described above, a dosage unit refers to a unitary, i.e. a single dose which is capable of being administered to a patient, and which can be readily handled and packed, remaining as a physically and biologically stable unit dose comprising either the drug as such or a mixture of it with solid or liquid pharmaceutical diluents or carriers.

In certain embodiments, organisms are engineered to produce drugs identified according to the invention. These organisms can release the drug for harvesting or can be introduced directly to a patient. In another series of embodiments, cells can be utilized to serve as a carrier of the drugs identified according to the invention.

Where the drug is intended for use as a therapeutic to alleviate disease associated with the central nervous system (CNS) an additional administration problem can need to be addressed: overcoming the so-called “blood-brain barrier,” the brain capillary wall structure that effectively screens out all but selected categories of molecules present in the blood, preventing their passage into the brain. The blood-brain barrier can be bypassed effectively by direct infusion of the drug into the brain. In certain embodiments, however, the blood-barrier can be circumvented by using a drug expressed by an organism with an inherent ability to penetrate the blood-brain barrier, i.e., an organism with tissue-specificity for the brain such as, for example, T. cruzi. Alternatively, an organism expressing a drug can be further genetically-modified to insure that the desired expression product is modified to enhance its transport across the blood-brain barrier. For example, truncated forms of the expression product can be most successful. Alternatively, a drug according to the invention can be modified to render it more lipophilic, or it can be conjugated to another molecule which is naturally transported across the barrier, using standard means known to those skilled in the art, as, for example, described in Pardridge, Endocrine Reviews: 7:314-330 (1986) and U.S. Pat. No. 4,801,575.

The pharmaceutical compositions can be included in a container, pack, or dispenser together with instructions for administration.

When an active compound of the instant invention is intended for administration to a plant host, the compound can be applied directly to the plant environment, for example, to the surface of leaves, buds, roots or floral parts. Alternatively, the compound can be used as a seed coating. The determination of an effective amount of the compound as required for a particular plant is within the skill of the art and will depend on such factors as the plant species, method of planting, and soil type. Determination requires only routine skill. It is contemplated that compositions containing compounds of the invention can be prepared by formulating such compounds with adjuvants, diluents, carriers, for example, to provide compositions in the form of filings/divided particulate solids, granules, pellets, wetable powders, dust, aqueous suspensions or dispersions, and emulsions. It is further contemplated to use such compounds in capsulated form, for example, the compound s can be encapsulated within polymer, gelatin, lipids or other formulation aids such as emulsifiers, surfactants wetting agents, antifoam agents and anti-freeze agents, can be incorporated into such compositions especially if such compositions will be stored for any period of time prior to use. Application of compositions containing compounds of the invention as the active agent can be carried out by conventional techniques.

When an active compound is intended for administration to an insect, standard methods such as, but not limited to, aerial dispersal are contemplated.

Drugs identified by a method of the invention also include the prodrug derivatives of the compounds. The term prodrug refers to a pharmacologically inactive (or partially inactive) derivative of a parent drug molecule that requires biotransformation, either spontaneous or enzymatic, within the organism to release the active drug. Prodrugs are variations or derivatives of the compounds of the invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention can be called single, double, triple, and so on, depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of drugs discovered according to this invention can be combined with other features herein taught to enhance bioavailability.

Drugs as identified by the methods described herein can be administered to individuals to treat (prophylactically or therapeutically) disorders. In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a drug as well as tailoring the dosage and/or therapeutic regimen of treatment with the drug.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitroflirans) and consumption of fava beans.

One pharmacogenomics approach to identifying genes that predict drug response, known as “a genome-wide association,” utilizes a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. A SNP is a common alteration that occurs in a single nucleotide base in a stretch of DNA. For example, a SNP can occur once per every 1000 bases of DNA. A SNP can be involved in a disease process, however, the vast majority can not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach,” can be utilized to identify genes that predict drug response. According to this method, if a gene that encodes a drug's target is known, all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification. Alternatively, a method termed the “gene expression profiling,” can be utilized to identify genes that predict drug response. For example, the gene expression of an animal dosed with a drug can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a drug identified according to the invention.

Certain embodiments of the invention are described in the following examples, which are not meant to be limiting.

### EXAMPLE 1

### Preparation of Tagged GPCR

Various GPCR vectors were prepared for the baculovirus expression system containing epitope tags that allowed for easier purification of the receptor using standard techniques known by those skilled in the art. The following is a representative method of preparing a tagged GPCR utilizing 6xHis, FLAG or GST as tags. However, alternative tags can be incorporated and constructs can be made using other procedures known to those skilled in the art. Alternative tags, can include, without limitation, V5, Xpress, c-myc, HA, CBD, and MBP. Tags can be incorporated at the N- and C-termini of a receptor. CC Chemokine Receptor 5 (“CCR5”) was the receptor that was selected for preparation and tagging. Although CCR5 was the GPCR that was chosen for this experiment, other GPCRs can be tagged using the method according to the present invention. For CCR5, tags at the C-terminus of the receptor were incorporated to determine the character of the receptor's ligand-binding properties at the N-terminus region. The construction of C-terminal 6xHis tagged FLAG-tagged, and GST-tagged constructs are provided below as examples.

The cDNA for CCR5 was obtained from Receptor Biology (Rockville, Md.), and sub-cloned using standard Polymerase Chain Reaction (PCR) techniques and primers to the 5′ and 3′ ends of the CCR5. Alternatively, CCR5 can be obtained from a cDNA library using PCR and primers to the 5′ and 3′ ends of the CCR5. To create a C-terminal tag, CCR5 was subcloned into an E. Coli vector (pET30a) with a C-terminal 6xHis tag. The C terminus was tagged in order to determine the character of the receptor's ligand-binding properties at the N-terminal region.

A construct was prepared and cloned using a second tag. A FLAG tag (pFLAG-CTC, Sigma, St. Louis, Mo.) was incorporated at the C-terminus of CCR5 by subcloning the CCR5 into pFLAG-CTC plasmid, then excising the CCR5 with the C-terminal FLAG tag and ligating into the digested baculovirus vector (pBluebac 4.5, Invitrogen, Carlsbad, Calif.).

A third tag, GST, was incorporated into the C-terminus of CCR5 by using PCR to generate the GST tag from an appropriate vector, then ligating the CCR5-GST construct into the digested baculovirus vector (SmaI/EcoRI) using standard procedures known to those skilled in the art. Thereafter, the PCR product (CCR5) was incorporated into an entry vector using LR clonase (the enzyme required for the initial homologous recombination step), and then transferred into the baculovirus GST-tag vector using BP clonase (the enzyme required for secondary homologous recombination) to make a vector for baculovirus expression containing the CCR5 with a C-terminal GST tag (or FLAG or 6xHis tag) without enterokinase or thrombin cleavage sites. The resulting construct was analyzed using both restriction digestion and sequencing, and then transfected into insect cells, such as, Sf9 (Pharmingen, San Diego, Calif.) for expression.

To express the CCR5 tagged with 6xHis, FLAG, or GST tag in insect cells, the pBlueBac vector containing CCR5 DNA insert was cotransfected with Bac-N-Blue DNA using cationic liposome mediated transfection using standard techniques known to those skilled in the art. CCR5 was inserted into the baculovirus genome by homologous recombination. Cells were monitored from 24 hours post-transfection to approximately 4 to 5 days. After about 72 hours, the transfection supernatant was assayed for recombinant plaques using a standard plaque assay. Cells which have the recombinant virus produced blue plaques when grown in the presence of X-gal (5-bromo-4-chloro-3-indoyl-β-D-galactoside). These plaques were then purified, and the isolate was verified by PCR for correctness of recombination using standard techniques. From this, a high-titer stock was generated and infection performed from this stock for expression work using techniques standard to those skilled in the art. Controls for transfection generally included cells only and transfer vector without insert in cells.

In a related experiment, time courses with three GPCRs (CCR5, CXCR4 and Gonadotropin Releasing Hormone Receptor (GnRHR)) demonstrated that less time is required for maximum expression of the receptor using Gateway adapted systems (Invitrogen, Carlsbad, Calif.) than for standard baculovirus systems. Also, the time courses demonstrated less proteolysis of the protein. For example, with CCR5, CXCR4 and GnRHR, expression required approximately 24-72 hours. Furthermore, less proteolysis resulted for GnRHR and CCR5.

Sf9 cells (and High Five insect cells, Invitrogen, Carlsbad, Calif.) were maintained both as adherent and suspension cultures using standard techniques known to those skilled in the art. The adherent cells were grown to confluence and passaged using the sloughing technique at a ratio of 1 to 5. Suspension cells were maintained in spinner flasks with 0.1% pluronic F-68 in order to minimize shearing for approximately 2 to 3 months by sub-culturing to a density of 1×10⁶ cells/ml.

After the CCR5 having either the GST, 6xHis or FLAG tag was expressed, a time course after infection with recombinant virus was used to define optimal growth conditions for expression using standard techniques. Aliquots of cells from spinner flasks were taken for this time course, centrifuged at 800×g for 10 minutes at 4° C. and both supernatant and pellet were assayed by SDS-PAGE/Western blot analysis. CCR5 was expected to be in the membrane fraction (pellet). In order to confirm the presence of the CCR5 in the membrane fraction, the membrane fraction was separated from the soluble fraction (cytosolic) using centrifugation. The portion of the membrane fraction was run on SDS-PAGE and transferred to nitrocellulose by western blotting. To provide functional conformation to CCR5, solubilization was conducted in a buffer, which included NP-40 (or Nonidet P-40, Sigma, Mo.) and low magnesium and calcium concentrations and no NaCl concentration.

Various methods can be used for immobilizing the tagged receptor. Such methods are generally known to those skilled in the art. To immobilize the receptor tagged with 6xHis, nickel chelation was used. To immobilize the receptor tagged with the FLAG tag, M2 antibody affinity column coupled to agarose was used. Also, to immobilize the GST tagged receptor, gluthathione sepharose (or agarose) was used. Thereafter, the receptor was detected using an antibody of the tag. For example, for 6xHis, an anti-His antibody was used. For FLAG, an M1 FLAG (N-terminal) or M2 antibodies (C-terminal) was used. For GST, an anti-GST antibody was used. Other detection methods are commonly known to those skilled in the art.

### EXAMPLE 2

### Isolation of Ligand from a Translated cDNA Source

The following experiment was designed to determine if affinity de-orphaning when practiced according to the methods of the present invention, utilizing tissue or cell fractionation, was successful for de-orphaning of GPCRs. A GPCR having a known ligand, CCR5, was used so that the conditions can be optimized and generalized to other receptors. Although CCR5 has a known ligand, the role and function that CCR5 plays in the human body is not entirely known. Also, even though the following example discusses the de-orphaning of CCR5, methods according to the invention is appropriate for any GPCR. For purposes of this experiment, using a GPCR with a known ligand allowed for the careful analysis of every step to optimize the process used for de-orphaning. Also, the following example was performed to demonstrate the power and utility of these methods according to the invention that involves the use of affinity de-orphaning by utilizing cDNA small pool libraries for the ligand source.

CCR5 was obtained and prepared with a GST tag according to the steps provided in Example 1. CCR5-GST was immobilized on a glutathione column using the GST tag, and incubated with ligands obtained using in vitro translation of small pools of approximately 100 clones from a cDNA library. CCR5 was immobilized in functional form and used to isolate RANTES, a natural ligand for CCR5, which was obtained ATCC (Manassas, Va.) from its IMAGE collection. Thereafter, a human lung cDNA library was spiked with RANTES and was transcribed and translated in vitro using the TNT Quick Coupled Wheat Germ kit, available from Promega Corporation (Madison, Wis.) with [³⁵S]-Met. RANTES cDNA alone was used as a positive control for translation and binding experiments.

A human lung cDNA library was chosen because RANTES was originally isolated from a human lung library. Also, libraries derived from tissues having an abundant number of immune cells, such as spleen and thymus tissues also are possible sources. The cDNA was in a bacterial host (E. coli). In general, the clones were transcribed and translated, and a mixture of their respective proteins was produced in a concentrated sample. This sample then was then probed with a receptor (i.e., CCR5) to determine if there are any proteins in the pool that can bind to the receptor of interest. The concentration was approximately 5×10⁹ colony forming units per ml. Thus, dilution was necessary to dilute the number of clones to approximately 100 per pool. After dilution and without amplification, approximately 100 clones were pipetted onto each of 100 LB amp agar plates and grown overnight at 37° C. or until the colonies were approximately 1 mm in diameter.

To harvest the pools, 1 to 2 ml of LB amp media was applied to the agar plate and the colonies were scraped into the liquid which was then transferred to a culture tube and incubated with shaking at 37° C. for approximately 1-2 hours. Half of the culture was saved for a glycerol stock to use for sequencing and half was used to perform a cDNA mini-prep to provide cDNA for in vitro translation. Mini-preps were performed for each pool to isolate the cDNA from the pools. These pools were submitted for in vitro translation using Promega's TNT Coupled Wheat Germ Extract system, namely, each pool was placed in a well of a 96-well tissue culture plate. The cDNA from the small pool libraries was added to the wheat germ extract, along with buffer, polymerase, amino acid mixture, radiolabeled amino acid (typically [³⁵S-Met], RNase inhibitor, and nuclease-free water provided by Promega (Madison, Wis.).

The reaction mixture was incubated at 30° C. for 2 hours to transcribe and translate the cDNA to mRNA and protein in vitro. The solubilized, immobilized receptor on a support (beads, for example) was added to the mix of translation products in each well, and the translation mixture was incubated at room temperature for approximately 20-30 minutes and the placed on ice for 10 minutes. The receptor was immobilized on a support, such as a bead, in order to wash out the non-specific binders from the translation mixture of translation products.

After binding was allowed to occur, the receptor-bead complex was filtered and washed with buffer with salt (50 mM PIPES, pH 7.5, 3 mM calcium chloride, 15 mM magnesium chloride, 0.1% NP-40; 250 mM NaCl) to remove nonspecific binders. Subsequent to washing, SDS-PAGE sample buffer was added directly to the immobilized receptor with potential bound ligands, boiled 1 minute (thereby separating bound ligand from the support), and loaded directly onto an SDS-PAGE gel for separation by size using electrophoresis.

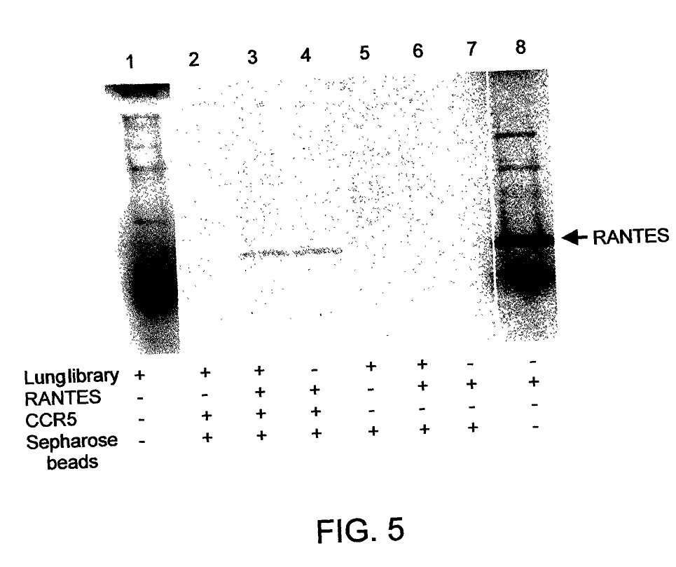

After electrophoresis, the gel was dried and exposed to phosphorimage plates for approximately 2-4 hours for detection of any radioactive bands. Standards were used for comparison and controls were performed for non-specific binding. FIG. 5 shows the results of this affinity de-orphaning using cDNA. For example, as demonstrated in FIG. 5, Lane 1 is the translation of the human lung library, namely including only the cDNA from the human lung library. Lane 2 is the incubation of the lung library with immobilized CCR5, namely including the cDNA from the human lung library with CCR5 immobilized on sepharose beads. Lane 3 is the incubation of the human lung library containing RANTES with immobilized CCR5, namely including the cDNA from the human lung library, RANTES, and CCR5 immobilized on sepharose beads. Lane 4 is a control for RANTES binding without the presence of other proteins from the human lung library, namely including only RANTES and CCR5 immobilized on sepharose beads. Lane 5 is a control for binding of the human lung library to a support only without CCR5, namely including the cDNA from the human lung library and sepharose beads. Lane 6 is a control for binding of lung library containing RANTES to a support only, without CCR5, namely including the cDNA from the human lung library, RANTES and sepharose beads. Lane 7 is a control for RANTES only binding to a support only without CCR5, namely including RANTES and sepharose beads. And, lane 8 is a standard for RANTES, namely including only RANTES. The lanes containing RANTES demonstrate that binding was observed for this pool, as demonstrated in FIG. 5. For example, binding was exhibited only where RANTES was included with the CCR5 receptor, aside from Lane 8, which was the control. Accordingly, CCR5 captured RANTES which was translated from the cDNA library, demonstrating that binding was observed, which indicates that the method of utilizing solubilized, immobilized CCR5 was successful.

In a further protocol, the method is repeated to identify pool(s), which are a subset of the initial pool, containing the ligand seen in the initial pool. This method is repeated with smaller subsets of the initial pool. Accordingly, any pool (e.g., containing approximately 100 clones) that had a ligand binding, as determined by SDS-PAGE, is further split into about 10 pools of about 10 clones to identify which of these pool(s) contain a subset of clones from the initial pool of 100 clones, contain molecules that will bind CCR5. For those positive pools of about 10 clones, the pools are further split until a pool (or pools) containing a single clone that translates to a molecule that will bind CCR5 is identified. After the single clone is identified, the clone is sequenced for identification. Also, once the single clone is isolated, the clone is grown in its bacterial host, mini-preps of the plasmid DNA containing the specific clone of interest is prepared, and a small amount of the plasmid DNA is sent for sequencing using standard dye terminator cycle sequencing methods (DNA sequencing performed by a core DNA sequencing facility such as Veritas (Rockville, Md.) or Tufts University (Boston, Mass.)).

### EXAMPLE 3

### Isolation of Ligand from a Tissue Cell Culture Extract

The experiment described in Example 2 was repeated in a proof-of-principle fashion, i.e., to de-orphan CCR5 from tissue cell culture extracts using the methods of the present invention as if it was unknown that RANTES was the natural ligand. The following experiment was designed to confirm that affinity de-orphaning utilizing tissue or cell fractionation was successful for de-orphaning GPCRs. Although the following example discusses the de-orphaning of CCR5, methods according to the invention are appropriate for any GPCR. For purposes of this experiment, using a GPCR with a known ligand allowed for the careful analysis of every step to optimize the process used for de-orphaning. Also, the following example was performed to demonstrate the power and utility of these methods according to the invention that involves the use of affinity de-orphaning by utilizing tissue cell culture extract for the ligand source.

Expressed CCR5-GST was immobilized in the same fashion as described above using a glutathione-sepharose resin. THP-1 cells, a monocytic cell line, were induced with lipopolysaccharide (LPS) from E. coli extracts (Sigma, St. Louis, Mo.), and grown for 48 hours. THP-1 cells were selected for the purpose of this experiment because THP-1 cells are a general monocytic cell line that expresses CCR5 (the receptor of interest). Also, THP-1 cells, and other monocytes are involved in the inflammatory process and migrate to sites of inflammation in response to a chemokine gradient. These cells can also be induced to produce more chemokines by using reagents such as lipopolysaccharide (LPS) from E. coli.

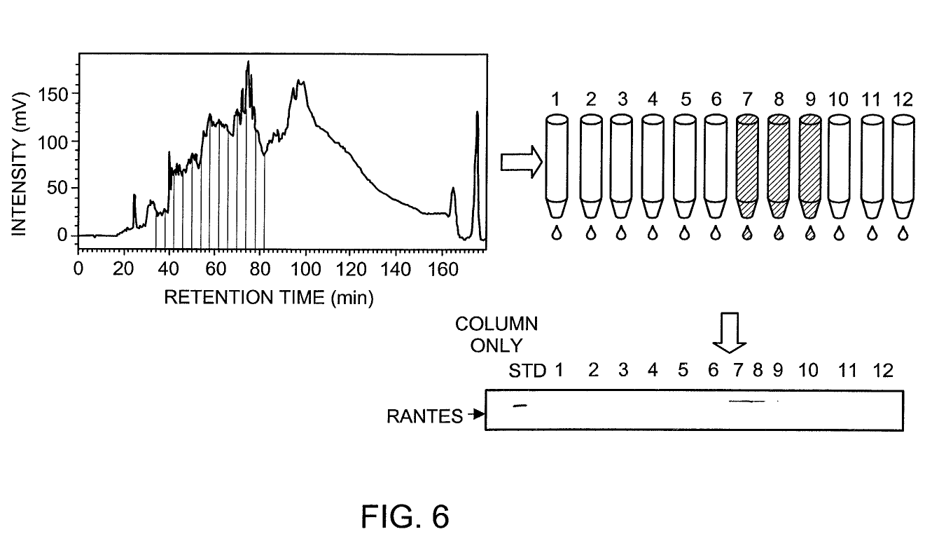

The THP-1 cells were harvested by centrifugation. In this case, the supernatant was retained because a chemokine or a small-secreted protein was being located. The supernatant was put directly onto a RP-HPLC (reverse phase high performance liquid chromatography) column and a crude fractionation was performed (FIG. 6). The crude fractionation of HPLC of concentrated THP-1 culture supernatant yielded pools containing hundreds of proteins, as demonstrated in FIG. 6. Each fraction (labeled 1-12) contained hundreds of proteins totaling over 3 mg each. The quantity of RANTES quantitatively determined to be approximately 1 μg in approximately 2-3 L. These crude fractions or pools were purified by CCR5 affinity columns and by incubating with the immobilized CCR5. Non-specific binders were washed away, and the CCR5 with any bound ligand was loaded directly onto an SDS-PAGE gel (after boiling in SDS-PAGE sample buffer to remove the support). The gel was developed with silver stain. Lanes 1-12 on the gel correspond to fractions 1-12.

The CCR5 with column fractions were compared to CCR5-only controls because there are proteins that non-specifically bind to the column, and because the CCR5 receptor and any contaminants present in the preparation can be seen as protein bands. Therefore, samples were compared such that only the ligands that bound specifically to CCR5 were chosen for sequencing. Specifically, Lanes 7, 8 and 9 showed bands indicating that ligands bound to CCR5. Therefore, bands that were detected only in the fraction samples were submitted for sequencing and identified by mass spectrometry. The mass spectrometry of silver stained gels identified RANTES as a ligand for CCR5. The mass spectrometry also identified 6 additional binders. Confirmation was performed by western blotting using an anti-RANTES antibody. Specifically, confirmation of the identity of RANTES was done by first comparing the standard on the gel then by western blotting with a specific antibody, and finally by sequencing.

Thus, RANTES was identified from a cell supernatant fractionation in two steps: HPLC fractionation followed by affinity purification using immobilized CCR5. Two of the binders were identified from this experiment and have been characterized for their specific function with respect to CCR5. The first binder is a histone, such as, for example, H3, H4, and H2A and, the second, is statherin. Preliminary data for a statherin-like peptide demonstrates calcium signaling in CCR5-containing cells (THP-1), indicating this can be a truly previously unidentified ligand for CCR5.

### EXAMPLE 4

### CCR5 Binding to a Ligand

The following experiment was performed to demonstrate further the power and utility of the methods according to the invention. The method exemplified in this example involves the use of immobilized receptors to first, isolate a ligand, and second, isolate the CCR5 ligand specifically.

CCR5 was obtained and prepared with a GST tag bound to glutathione column according to the steps provided in Example 1. Thereafter, a ligand mixture (RANTES, MDC, gonadotropin releasing hormone) was incubated with immobilized CCR5 to determine which ligands bound to the receptor. After washing to remove non-specific binding, the SDS-PAGE sample buffer was added to each, boiled for 1 minute, and loaded onto an SDS-PAGE gel. In order to analyze the beads, SDS-PAGE was performed on the various samples and transferred to nitrocellulose by western blotting to determine which ligands were bound. Duplicate samples were run, one group for detection with anti-RANTES antibody and one group for detection with anti-MDC antibody.

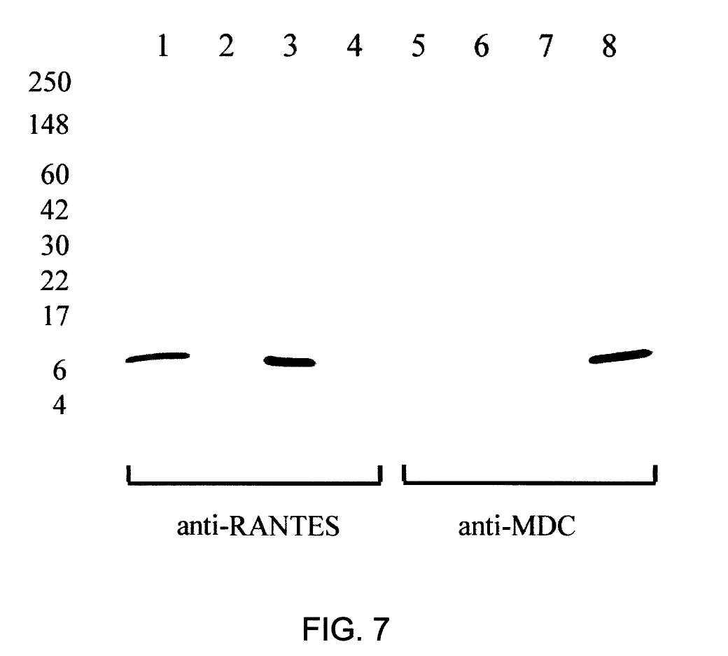

FIG. 7 shows the binding observed in the experiment. The molecular weights (in kDa) as determined by the standards are listed on the left side of the blot. The individual lanes are numbered across the top of the blot. Lanes 1-4 were developed with anti-RANTES antibody. Lane 1, shows what is detected by anti-RANTES antibody after binding to their receptor. The band at approximately 8 kDa is RANTES. Lane 2 was a control binding experiment having the ligands but without a receptor, thereby demonstrating that RANTES specifically binds to the CCR5 receptor. Lane 3 is a RANTES standard (positive control). Lane 4 is the MDC (macrophage-derived chemokine) standard (negative control—CXCR4 ligand). Lanes 5-8 were developed with anti-MDC (macrophage-derived chemokine, a CCR4 ligand ) antibody. Lane 5 is the same experiment as in Lane 1 but developed with anti-MDC antibody demonstrating that the CCR4 ligand MDC does not bind to CCR5, demonstrating specificity. Lane 6 is the same as lane 2 but developed with anti-MDC antibody. Lane 7 is RANTES standard and Lane 8 is MDC standard. The functionality of the immobilized CCR5 was shown, for example, in FIG. 2A.

The western blot shown in FIG. 7 demonstrates that RANTES, the CCR5 ligand, bound specifically to CCR5 as seen in Lane 1. Furthermore, Lane 2, the control lane having the ligands without the receptor, exhibited no binding, as expected. The experiment confirmed that the use of immobilized receptors was able to isolate a ligand. Moreover, the immobilized receptors accomplished isolating the CCR5 ligand specifically.

### EXAMPLE 5

### Natural Ligand Binding to an Orphan GPCR (C5L2)

The following experiment involves the use of the methods of the present invention for de-orphaning a GPCR, namely C5L2, an orphan receptor with approximately 40% identity to C5a receptor, approximately 33% identity to C3a receptor, approximately 29% identity to fMLP receptor, and approximately 25% identity to drosophila galanin receptor. The experiment demonstrates that the use of immobilized receptors enables the isolation of ligands and, more specifically, the natural ligand for the C5L2.

A genomic DNA library was obtained from ClonTech (Palo Alto, Calif.). From this library, C5L2 was isolated using PCR and primers to the 5′ and 3′ ends of C5L2. The C5L2 was tagged at the C-terminus with a GST tag. In order to make a C-terminal GST-tagged receptor, Gateway adaptor primers were used to add sequences to permit homologous recombination with a vector adapted for this purpose as described in Example 1, with a GST tag at the 3′ end.

To express the C5L2 with a GST tag in Sf9 cells from Invitrogen (Carlsbad, Calif.), the pBlueBac vector containing C5L2 DNA insert was cotransfected with Bac-N-Blue DNA using cationic liposome mediated transfection using standard techniques known to those skilled in the art. CCR5 was inserted into the baculovirus genome by homologous recombination. Cells were monitored from 24 hours post-transfection for approximately 4 to 5 days. After about 72 hours, the transfection supernatant was assayed for recombinant plaques using a standard plaque assay. Cells which have the recombinant virus produced blue plaques when grown in the presence of X-gal (5-bromo-4-chloro-3-indoyl-β-D-galactoside). These plaques were then purified, and the isolate was verified by PCR for correctness of recombination using standard techniques. From this, a high-titer stock was generated and infection performed from this stock for expression work using techniques standard to those skilled in the art. Controls for transfection included cells only and transfer vector without insert in cells.

Sf9 cells (and High Five insect cells) were maintained both as adherent and suspension cultures using standard techniques known to those skilled in the art. The adherent cells were grown to confluence and passaged using the sloughing technique at a ratio of 1 to 5. Suspension cells were maintained in spinner flasks with 0.1% pluronic F-68 in order to minimize shearing for approximately 2 to 3 months by sub-culturing to a density of 1×10⁶ cells/ml. Virus stocks were prepared in Sf9 cells and protein production was done in High Five cells.

After the C5L2 having the GST tag was expressed, a time course after infection with recombinant virus was used to define optimal growth conditions for expression using standard techniques. Aliquots of cells from spinner flasks were taken for this time course, centrifuged at 800×g for 10 minutes at 4° C. and both supernatant and pellet were assayed by SDS-PAGE/Western blot analysis. C5L2 was expected to be in the membrane fraction (pellet). In order to confirm the presence of the C5L2 in the membrane fraction, the membrane fraction was separated from the soluble fraction (cytosolic) using centrifugation. The membrane fraction was run out on a SDS-PAGE gel and transferred to nitrocellulose by western blotting. To provide functional conformation to C5L2, solubilization was conducted in a buffer, which included NP-40 (or Nonidet P-40, Sigma, Mo.) and low magnesium and calcium concentrations and no NaCl.

The GST tagged receptor was immobilized on a gluthathione sepharose support. The receptor was detected using an anti-GST antibody.

THP-1 cells, a monocytic cell line, were induced with lipopolysaccharide (LPS) from E. coli extracts (Sigma, St. Louis, Mo.), and grown for 48 hours. THP-1 cells were selected for the purpose of this experiment because THP-1 cells are a general monocytic cell line that expresses chemokine receptors and many chemokines (a class similar to the C5L2 receptor). Also, THP-1 cells, and other mono cytes are involved in the inflammatory process and migrate to sites of inflammation in response to a chemokine gradient. These cells also can be induced to produce more chemokines by using reagents such as lipopolysaccharide (LPS) from E. coli.

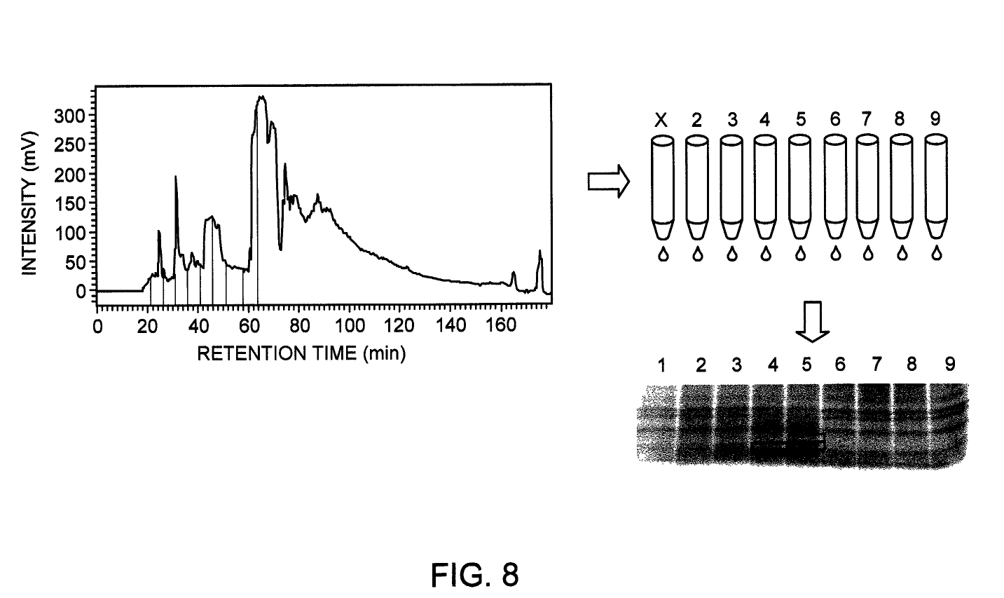

The THP-1 cells were harvested by centrifugation. In this case, the supernatant was retained because a chemokine or a small-secreted protein was being located. The supernatant was put directly onto a RP-HPLC (reverse phase high performance liquid chromatography) column and a crude fractionation was performed. The results are shown in FIG. 8. The crude fractionation of HPLC of concentrated THP-1 culture supernatant yielded pools containing hundreds of proteins. Each fraction (test tubes labeled 2-9) contained hundreds of proteins. These crude fractions or pools were purified with C5L2 affinity columns. The test tube marked X in FIG. 8 was the control test tube, which only contained the C5L2 receptor bound to the support in buffer containing 0.1% NP40, 20 mM PIPES, 3 mM calcium chloride and 15 mM magnesium chloride. More particularly, these fractions (test tubes 2-9) were incubated with the immobilized C5L2 receptor. Non-specific binders were washed away, the C5L2-support-binding molecule complex was boiled in SDS-PAGE sample buffer to remove the support, and the C5L2 with any bound ligand was loaded directly onto an SDS-PAGE gel. The gel then was developed with silver stain.

The C5L2 with column fractions were compared to C5L2-only controls because there are proteins that non-specifically bind to the column, and because the C5L2 receptor and any contaminants present in the preparation can be seen as protein bands. Therefore, samples were compared such that only the ligands that bound specifically to C5L2 were chosen for sequencing. As provided in FIG. 8, Lane 1 is the C5L2-only control showing the background for the experiment. Lanes 2 through 9 show the materials bound to the immobilized C5L2 receptor after fractions 2 through 9, respectively, are interacted with the C5L2 receptor. The proteins that uniquely bind to the C5L2 receptor in this experiment are highlighted with the boxes in Lanes 4 and 5. Accordingly, Lanes 4 and 5 show bands indicating that certain natural ligands bound to the immobilized C5L2 receptor. The bands from Lanes 4 and 5 were cut from the gel and were submitted for sequencing and identification by mass spectrometry.

The experiment confirms that methods of the invention can successfully isolate a natural ligand for an orphan GPCR receptor (the C5L2 receptor) from a naturally-derived source, particularly when that receptor is solubilized and immobilized.

### EXAMPLE 6

### Protocol for De-orphaning a GPCR of Interest

Certain approaches according to the invention can be used when de-orphaning a GPCR in order to identify and isolate potential lead compounds that can be used, for example, in drug discovery. Generally, the steps involved in de-orphaning a GPCR of interest vary depending on the particular resources used. The following describes general methods and considerations according to the invention, and is provided for example purposes.

If a GPCR of interest, for example, an orphan GPCR has been identified, then the following steps are taken. The GPCR of interest is first isolated from a cDNA library using standard PCR techniques and primers to the 5′ and 3′ ends of the gene of interest. If, however, the GPCR of interest has already been isolated, then the GPCR only needs to be subcloned into prepared vectors for expression with an appropriate tag and for the appropriate system. Also, a second round of PCR is used to add Gateway adaptors as provided by Invitrogen (Carlsbad, Calif.) to the ends of the GPCR clone such that it can be incorporated directly into a Gateway vector (Invitrogen, Carlsbad, Calif.) directionally. Based upon the Gateway terminology provided by Invitrogen, this vector is considered an homologous recombination vector. Next, the homologous recombination vector is then transferred to the prepared custom-designed Gateway vector adapted to have a C-terminal tag for a baculovirus system using a simple homologous recombination reaction.

Thereafter, this vector is used to infect a cell for isolation, such as, insect cells (Sf9 or High Five insect cells, for example) to first isolate a single virus clone. After the isolation of the single virus clone, a large virus stock is prepared for future production of the GPCR of interest. As described previously, cells having the recombinant virus produces blue plaques when grown in the presence of X-gal (5-bromo -4-chloro-3-indoyl-β-D-galacto side). These plaques can be purified. Also, the isolates are checked by PCR for correctness of recombination, using standard techniques known to those skilled in the art.

Infection of Sf9 (or High Five) insect cells is accomplished by adding the virus stock to the cells and incubating for approximately 24 to 72 hours. The cells are harvested at the time for optimal expression of the GPCR of interest. This expression level is determined using an antibody to the tag, for example GST, and standard western blotting procedures with the infected cells. Once expression is confirmed and optimized for yield, the GPCR is be produced in large quantities in the insect cells (Sf9 or High Five). Cells containing the receptor of interest are harvested by centrifugation (800×g for 10 minutes at 4° C.). Lysis of the cells is performed using a hypotonic solution (e.g., Tris or PIPES buffer, pH 7.5 with protease inhibitor cocktail and detergent) and a hand-held tissue homogenizer. Following lysis, centrifugation of cellular debris away from the other cellular material is performed.

Solubilization of the receptor (and other membrane proteins) is performed using a buffer containing NP-40 and protease inhibitors in the absence of NaCl, and homogenized using, for example, a hand-held tissue homogenizer. This solution is rotated gently for approximately 1 hour at 4° C. to maximize solubilization of the receptor. Similarly, the tagged GPCR is immobilized by gently rotating the solubilized solution with buffer-equilibrated support, or glutathione-sepharose beads for 1 hour at 4° C. Generally, methods according to the invention are used with any support known to those skilled in the art. Examples of such supports include, but are not limited to, any matrix, any resin, any bead or any column. Specific supports include, but are not limited to, glutathione-sepharose beads, sepharoses, agaroses including, for example, M2 FLAG, antibody resin, nickel columns, nitrocellulose or similar 2-dimensional matrices (including PVDF (polyvinylidene fluoride) membrane, and glass slides. The non-specifically bound proteins from the cellular preparation are removed, for example, by gently but exhaustively washing in with buffer.

Following this, the GPCR is ready to mix with the source of potential ligands of the GPCR, such as, with fractions of tissue, cellular extracts or translated cDNA pools. Any naturally-derived source can be used, but it is beneficial to rationally choose certain sources relevant to the GPCR under consideration. To choose an appropriate tissue, cellular, or cDNA source for potential ligands of the GPCR, many parameters are taken into account in order to isolate the GPCR ligand of an orphan GPCR. First, for example, the sequence homology of the orphan receptor to all known (and orphan) receptors can be determined. The sequence homology can put the orphan receptor into a specific class or an entirely new class. If the class is known, then the ligands are expected to be similar or from similar sources. The sequence homology can also associate the orphan receptor to a limited number of tissues or cell types. In addition, the sequence homology can indicate that an orphan is likely to bind, for example, a peptide, protein, nucleotide, and a small biogenic amine.

For potential peptide or protein ligands, an approach utilizing cDNA libraries can be used. However, for small molecules of any type or any non-proteinaceous ligand types, the tissue or cellular approach can be more effective than using cDNA libraries. For a receptor that has sequence homology to a chemokine or chemoattractant receptor (or similar receptors), another approach can be used. Chemokine or chemoattractant receptors (and related receptors) are found on a cell and respond to ligands in a distant site. The chemokine or chemoattractant receptor response causes migration of the cell to that distant site.

The chemokine or chemoattractant receptor response can be positive, such as, for example, a response to fight infection and/or inflammation. Likewise, the response can be negative, such as, for example, the metastasis of tumor cells. For receptors such as these, the information from known systems aids in the identification of potential distant sites. In addition, since the ligands of chemokine or chemoattractant receptors are generally proteinaceous in nature, the use of cDNA library provides significant advantages, such as, for example, allowing many more potential ligands to be screened.

Determining the tissue expression pattern of the receptor can also limit the tissue, cellular, or cDNA library choice for potential ligands of a GPCR. For example, if a receptor is only expressed in a certain region of the brain, it is likely that this region of the brain is a tissue to use for a ligand source. FIG. 3, as described in more detail herein, provides a general exemplary diagram outlining the basic considerations for identifying a ligand source for testing. Any prior GPCR or GPCR ligand information, such as a tissue expression pattern, will likely shorten the amount of time and work involved in deciding the most appropriate approaches in the methods according to the invention.

After selecting the source of a ligand for a GPCR under consideration, the next step is to identify the ligand. An immobilized receptor, prepared as described above, is mixed with a potential ligand source, in this case either tissue or cellular fractions and translated cDNA pools, separately. Generally, each of these binding mixtures is incubated at 4° C. and washed to remove non-specific binders. All samples is run on SDS-PAGE. The binders from tissue or cellular fractions is detected using silver stain and identified directly from the cut-out protein band using mass spectrometric sequencing. Optionally, the identification is determined in conjunction with database identification. The binders from the translated cDNA library pools are detected using radioactivity and phosphorimaging. Also, pools with a positive binder are subfractionated or diluted into successively smaller pools (for example, a pool having 100 clones that exhibited ligand binding, as determined by SDS-PAGE, is further split into 10 pools of 10 clones and so on) until a single binding ligand is identified. Next, the cDNA can be prepared using standard techniques to make plasmid mini-prep cDNA for sequencing using standard dye terminator cycle sequencing methods (DNA sequencing performed by a core DNA sequencing facility such as Veritas (Rockville, Md.) or Tufts University (Boston, Mass.)).

After identification of the ligand for a target GPCR, confirmation or validation is conducted, if desired. Confirmation or validation is accomplished by synthesizing the target if the GPCR of interest is a small molecule or peptide. If the target GPCR is a protein, confirmation or validation is accomplished by expressing the receptor. Optionally, if synthesis or expression is not possible, confirmation or validation is accomplished by isolation of the ligand in larger quantities. Subsequently, the ligand binding is confirmed in a purified fashion (e.g., pure ligand can be incubated in the same fashion as before for initial confirmation). Furthermore, a functional assay is performed. In one example of performing a functional assay, the receptor is co-expressed in a mammalian system with a promiscuous G-protein followed by the performance of a calcium mobilization assay. Alternatively, the ligand is radiolabeled and a standard binding assay is performed as described above. Affinity of the ligand as demonstrated by either of the described assays is sufficient to confirm initial identification of a ligand to a GPCR.

The results from the assays can be provided a graphical representation of the activity of the peptide or protein of the ligand for the receptor. In addition, subsequent confirmation studies can be conducted to further validate or confirm a ligand of a GPCR of interest. Furthermore, the ligand can be synthesized, altered or modified as appropriate for therapeutic, pharmaceutical, and/or diagnostic applications.

### EXAMPLE 7

### Cloning, Tagging, Expressing, Solubilizing, and Immobilizing a GPCR

A. General Methods

Various GPCR vectors can be prepared for the baculovirus expression system containing epitope tags using standard techniques known by those skilled in the art that allowed for easier purification of the receptor. Tags may be incorporated at the N- or C-terminus of proteins. For GPCRs, tags at the C-terminus of the receptor can be incorporated to determine the character of the receptor's ligand-binding properties that are in the N-terminal region of the molecule. The construction of a C-terminal 6xHis tagged and C-terminal FLAG construct are given below as examples. Alternative tags may include, for example, GST, V5, Xpress, c-myc, HA, CBD, and MBP. These constructs can be made by analogous procedures using standard techniques known by those skilled in the art.

The 6xHis tag enables a one-step purification using nickel chelation. The cDNA for a GPCR can be isolated from an appropriate cDNA library using Polymerase Chain Reaction (“PCR”) and primers for the 3′ and 5′ ends of the desired gene, as well as the middle of the gene. To create a C-terminal tag, the gene of interest is subcloned into an E. coli vector, pET30a, with a C-terminal 6xHis tag. The newly created receptor is then excised and ligated into pBlueBac, a baculovirus transfer vector (Invitrogen, Carlsbad, Calif.). The construct is analyzed using both restriction digestion and sequencing, and then transfected into Sf9 insect cells (Pharmingen, San Diego, Calif.) for expression as typically done by those skilled in the art of protein expression.

A C-terminal bacterial FLAG construct can be obtained from Sigma (pFLAG-CTC). A similar strategy using standard techniques can be employed for the construction of this vector. GPCR is subcloned into the pFLAG-CTC plasmid, then excised with the C-terminal FLAG tag and ligated into the digested pBlueBac vector. The construct is analyzed using both restriction digestion and sequencing, and transfected into Sf9 or High Five insect cells for expression.

To express the GPCR gene in Sf9 or High Five cells, the pBlueBac vector containing the GPCR insert can be cotransfected with Bac-N-Blue DNA using cationic liposome mediated transfection using standard techniques. The GPCR is inserted into the baculovirus genome by homologous recombination. Cells are monitored from 24 hours post-transfection to 4-5 days. After about 72 hours, the transfection supernatant is assayed for recombinant plaques using a standard plaque assay. Cells which have the recombinant virus will produce blue plaques when grown in the presence of X-gal (5-bromo-4-chloro-3-indoyl-β-D-galactoside). These plaques are then purified and the isolate verified by PCR for correctness of recombination using standard techniques. From this, a high-titer stock is generated and infection performed from this stock for expression work using standard techniques. Controls for transfection include cells only and transfer vector.

A construct for the expression of a GPCR can be made from the starting vector pBlueBac 4.5 (Invitrogen) to remove the thrombin and enterokinase cleavage sites in the previously described vectors. The GST tag is added into the multiple cloning site by using PCR to generate the GST tag, then ligating into the digested vector (SmaI/EcoRI) using standard procedures known to those skilled in the art. Thereafter, the vector is made compatible with the Gateway technology from Invitrogen for ease of manipulation. This is accomplished by ligating into the SmaI site the cassette containing the recombination sites required for this technology (obtained from Invitrogen). The GPCR of interest is amplified using PCR with primers to extend the gene to contain the attachment sites for recombination. Then, the PCR product is incorporated into the baculovirus vector using BP clonase (the enzyme required for homologous recombination) to make a vector for baculovirus expression containing the GPCR with a C-terminal GST tag without the enterokinase or thrombin cleavage sites. This vector is cotransfected into Sf9 cells for preparation of the virus stock necessary for expression. The virus is plaque purified, and a PCR and sequence checked clone can be used for expression of the GPCR. Time courses with this construct with three GPCRs has demonstrated that less time is required for maximal expression of the receptor and proteolysis of the protein is less. For example, for CCR5, CXCR4 and GnRHR, 24-48 hours were required for expression. Less proteolysis resulted for GnRHR and CCR5.

To express the GPCR tagged receptor in Sf9 or High Five cells, the pBlueBac vector containing the tagged insert can be cotransfected with Bac-N-Blue DNA using cationic liposome mediated transfection using standard techniques. The tagged receptor is inserted into the baculovirus genome by homologous recombination. Cells are monitored from 24 hours posttransfection to 4-5 days. After about 24-72 hours, the transfection supernatant is assayed for recombinant plaques using a standard plaque assay. Cells which have the recombinant virus produces blue plaques when grown in the presence of X-gal (5-bromo-4-chloro-3-indoyl-β-D-galactoside). These plaques are purified and the isolate verified by PCR for correctness of recombination using standard techniques. From this, a high-titer stock can be generated and infection performed from this stock for expression work using standard techniques. Controls for transfection include cells and transfer vector.

Sf9 or High Five cells can be maintained both as adherent and suspension cultures using standard techniques known to those skilled in the art. The adherent cells can be grown to confluence and passaged using the sloughing technique at a ratio of 1:5. Suspension cells can be maintained in spinner flasks with 0.1% pluronic F-68 (to minimize shearing) for 2-3 months by sub-culturing to a density of 1×10⁶ cells/ml.

A time course after infection with recombinant virus can be used to define optimal growth conditions for expression using standard techniques. Aliquots of cells from spinner flasks are taken for this time course, centrifuged at 800×g for 10 minutes at 4° C. and both supernatant and pellet assayed by SDS-PAGE/Western blot analysis. The GPCR is expected to be in the membrane fraction (pellet). All viable systems are assayed in this fashion for levels of expression. Systems are assayed for activity using a standard radioligand binding assay on a membrane preparation using the natural ligand. If the natural ligand was not known as is often the case for orphan receptors (where the activity of the receptor has not been defined), the activity can be assayed for G protein-coupled signaling activity using standard cell-based assays known to those skilled in the art.

The membrane fraction is isolated by first pelleting the whole Sf9 cells (800×g for 10 minutes at 4° C.), then resuspending the pellet in a lysis buffer with homogenization. Typical lysis buffer is around neutral pH and contains a cocktail of protease inhibitors, both of which are standard techniques for those skilled in the art. For example, serine proteases, cysteine proteases, aspartyl proteases, and metalloproteases may be used with inhibitors, such as, for example, PMSF, aprotinin, leupeptin, phenathroline, benzamidine HCl. Membranes are pelleted. The solubilization of the receptor by different detergents (such as, but not limited to, β-dodecylmaltoside, n-octyl-glucoside, CHAPS, deoxycholate, NP-40, Triton X-100, Tween-20, digitonin, Zwittergents, CYMAL, lauroylsarcosine, etc.) is compared for quantity and activity. Solubilization may also be conducted using varying NaCl concentrations. Despite conventional thinking, the step of solubilization using low salt, for example, low calcium and magnesium concentrations and substantially in the absence of NaCl may provide unexpected optimal conditions for solubilization when compared for quantity and activity. Having 0.0 nM NaCl, although counter-intuitive, has been discovered to provide optimal conditions when solubilizing and immobilizing candidates with binding properties, such as, for example, CCR5 and CXCR4. A candidate for isolation is carried through for purification as described below.

After determining an appropriate detergent for solubilization and activity, such as, for example, NP-40, the GPCR is purified from the membrane fraction. The exact purification scheme also depends on the construct chosen, which is subject to activity and ease of solubilization. For purification of the 6xHis-tagged receptor, the membrane fraction is loaded onto a Ni-NTA column (Qiagen, Valencia, Calif.) in the presence of detergent, such as, for example, NP-40, washed extensively, and eluted with imidazole. Purification of the FLAG-tagged receptor is performed using the anti-FLAG M2 affinity matrix (Sigma, St. Louis, Mo.) in the presence of detergent and eluted with glycine. The purification is performed in the presence of an appropriate detergent, such as, for example, NP-40, found for the system in the experiment described herein. Activity of the purified receptor is assessed as described herein.

B. Methods Involving CCR5

1. Cloning and Expression of CCR5

CCR5 cDNA was obtained from Receptor Biology (Beltsville, Md.) in the vector pcDNA3. Tags were added to the C-terminus of the receptor for use in immobilizing them for affinity purification assays using standard techniques, as described herein. In addition, CCR5 cDNA correlating to GenBank Accession #U57840 may be used in the vector pcDNA3. The only difference between CCR5 cDNA from Receptor Biology and CCR5 cDNA correlating to GenBank Accession #U57840 is a point mutation at base 180 that changes Leucine to a Glutamine in the amino acid sequence. The following are specific examples from experiments using this tagging method.

Construction of CCR5 with C-Terminal Histidine Tag (Insect Select Expression System) The CCR5-HIS construct was derived from this system. PCR was performed using CCR5/pcDNA3 (from Receptor Biology) as the template and primers 5-Age His and 5-Spe His. The first primer introduced a unique Spe I site just before the initiator ATG of CCR5. The second primer mutated the stop codon of CCR5 into an Age I site in-frame with the histidine tag of pIZT/V5-His (Invitrogen, Carlsbad, Calif.). The PCR product was digested with Spe I and Age I, then ligated into similarly digested pIZT/V5-His. This construct is identified as CCR5-His-PIZT.

Construction of CCR5 with C-Terminal Histidine Tag (Baculovirus Expression System) CCR5-His-PIZT was digested with Hae II and Spe I, then filled in with Klenow fragment. The fragment containing CCR5-His was ligated into pBluebac 4.5 (Invitrogen) that was previously digested with Nhe I and blunted with Klenow. The construct was checked for correctness of orientation. This is called CCR5-HIS. The construct was confirmed by restriction digestion and sequencing using standard techniques. This construct has been used for expression and has been determined to be expressed sufficiently and in active form for use in the affinity purification screening.

Construction of CCR5 with C-Terminal FLAG Tag: PCR using standard techniques was performed using CCR5/pcDNA3 (from Receptor Biology) as the template and the primers 5-Xho pFLAG and 5-Sal pFLAG. The first primer engineered a unique Xho I site just before the initiator ATG of CCR5. The second primer mutated the stop codon of CCR5 into a Sal I restriction site (in-frame with the FLAG tag of pFLAG-CTC from Sigma). The PCR product was digested with Xho I and Sal I and ligated into similarly digested pFLAG-CTC (a bacterial expression vector). This construct is called CCR5-FLAG-CTC. CCR5-FLAG was then digested with Xho I and Sca I, and filled in with Klenow fragment. The fragment containing CCR5-FLAG-CTC was ligated into pBluebac 4.5 that was first digested with Nhe I then blunted with Klenow. This final construct is identified as CCR5-FLAG. The construct was confirmed by restriction digestion and sequencing using standard techniques. This construct has been used for expression and has been determined to be expressed sufficiently and in active form for use in affinity purification screening.

Construction of CCR5 with C-Terminal GST Tag: PCR was performed using CCR5/pcDNA3 (from Receptor Biology) as the template and primers GST-BamH1 and GST-Nde1. The first primer mutated the stop codon of CCR5 into a BamH I restriction site (in-frame with the FLAG/GST tag of pESP-3). The second primer introduced a unique Nde I site at the initiator codon of CCR5. The PCR product was digested with BamH I and Nde I and ligated into similarly digested pESP-3 (a yeast expression vector.) This construct is called pCP8. pCP8 was then digested with Nde I and Sma I, and filled in with Klenow fragment. The fragment containing CCR5-GST was ligated into pBluebac 4.5 that was first digested with Bgl II then blunted with Klenow. This final construct is identified as pCP10. When describing the protein, the construct is identified as CCR5-GST. The construct was confirmed by restriction digestion and sequencing using standard techniques. This construct has been used for expression and has been determined to be expressed sufficiently and in active form for use in affinity purification screening.

The vectors for the three new constructs (for CCR5-FLAG, CCR5-GST, and CCR5-HIS) were used to co-transfect Sf9 cells for the production of a viral stock of each. These viral stocks were purified using a standard plaque assay and then used in experiments to infect for the optimization of expression of CCR5 with its various C-terminal tags. High Five cells (Invitrogen) were also transfected with these CCR5 tagged constructs and tested for expression of CCR5. All constructs were determined to express the appropriately tagged receptor. Expression levels after 72 hours were as much as 5 times greater in High Five cells than those for Sf9 cells. All of the above described experiments were done using standard techniques known to those skilled in the art.

A fourth construct for the expression of CCR5 was made from the starting vector pBlueBac 4.5 (Invitrogen) to remove the thrombin and enterokinase cleavage sites in the previously described vectors. The GST tag was added into the multiple cloning site by using PCR to generate the GST tag, then ligating into the digested vector (SmaI/EcoRI) using standard procedures known to those skilled in the art. Next, the vector was made compatible with the Gateway technology from Invitrogen for ease of manipulation. This was done by ligating into the SmaI site the cassette containing the recombination sites required for this technology (from Invitrogen). CCR5 was amplified using PCR with primers to extend the gene to contain the attachment sites for recombination. Then, the PCR product was incorporated into the baculovirus vector using BP clonase (the enzyme required for homologous recombination) to make a vector for baculovirus expression containing CCR5 with a C-terminal GST tag without the enterokinase or thrombin cleavage sites. This vector was cotransfected into Sf9 cells for preparation of the virus stock necessary for expression. The virus was plaque purified, and a PCR and sequence checked clone was used for expression of CCR5. A time course with this construct showed that less proteolysis of the protein was observed and less time was necessary to obtain maximal expression of the receptor.

2. Activity of CCR5

Each of the tagged CCR5 genes (CCR5-GST, CCR5-FLAG, and CCR5-HIS) were expressed in Sf9 and High Five cells, as described in herein. Whole cells from Sf9 and High Five cell lines were lysed using hypotonic buffers (10 mM Tris, pH 7.4, 5 mM EDTA), and membrane preparations were made by homogenization and centrifugation using standard techniques known to those skilled in the art. Membrane preparations for CCR5-GST, CCR5-FLAG, and CCR5-HIS were assayed using a standard radioligand binding assay respectively. Binding assays were performed with 5 μg of membranes in 50 mM Hepes, pH 7.5, 1% BSA. The radioligand MIP1-β (obtained from New England Nuclear, “NEN”) was incubated with membranes at room temperature for 1 hour with and without cold MIP1α (a competing ligand; natural ligands for CCR5 are RANTES, MIP1β, and MIP1α), filtered, washed, and radioactive counts bound were detected using scintillation counting. Uninfected cells were used as a control for this experiment. The activity of the membrane preparations was comparable to that obtained by Receptor Biology (K_{d}<1 nM for MIP1-β binding) at least having 20% active protein.

3. Solubilization of CCR5

Both lysed whole cells and membrane preparations have been used for solubilization. Solubilization of the tagged versions of CCR5 (CCR5-FLAG, CCR5-GST, and CCR5-HIS) have been performed using many different combinations of detergents (such as, for example, NP-40, Triton X-100, β-D-maltoside, n-octylglucoside, CYMAL, Zwittergents, Tween-20, lysophosphatidyl choline, CHAPS, etc.,) salts (such as, for example, NaCl, CaCl₂, MgCl₂, MnCl₂, KCl, etc.,) buffers (such as, for example, Tris, Hepes, Hepps, Pipes, Mes, Mops, acetate, phosphate, imidazole, etc.,) at pH's ranging from about 6.8 to about 8.2. Conditions for optimal solubilization were found using Zwittergent 3-14 and low salt, e.g., low magnesium and calcium, but no NaCl (0.0 nM NaCl) at pH 8.1. In a preferred embodiment, at least 20% of the solubilized, immobilized protein is active. In highly preferred embodiments, at least 30%, 40%, 50% and 75% of the solubilized, immobilized protein is active.

4. Immobilization of CCR5

After solubilization, both CCR5-GST and CCR5-FLAG were immobilized onto affinity columns for purification and for use as active proteins. CCR5-GST was bound and immobilized onto glutathione-agarose (Pierce) and glutathione-sepharose (Amersham Pharmacia Biotech) and CCR5-FLAG was immobilized onto a specific antibody column that recognizes the FLAG epitope (M2 column, Sigma). The immobilization of the protein in active form required the use of detergents and appropriate pH and salt conditions to maintain activity while on the column. This activity was determined by radioactive binding using radiolabeled MIP-1β (as with the membrane assay above) and competition with cold MIP-1α. Uninfected cells have been used as controls for this activity, as well as the column alone. These experiments demonstrated the ability to immobilize microgram quantities of the receptor in pure form (sufficient for affinity purification screening) onto resin in active form.

C. Methods Involving CXCR4

1. Cloning and Expression of CXCR4

CXCR4 (a GPCR) was isolated from a spleen cDNA library in two halves and spliced together. These two fragments were isolated using PCR technology and primers to the 3′ and 5′ ends and the middle of the CXCR4 gene. A full-length clone was not isolated with the 3′ and 5′ primers; however, two halves were isolated and ligated together using a unique BamH I site in the gene. The identity of the construct was confirmed by sequencing. An alternate splice shorter form was also isolated, which is called CXCR4s. Tags were added to the C-terminus of the receptor for use in immobilizing them for affinity purification assays using standard techniques. The following are specific examples from experiments using this tagging method.

Construction of CXCR4 with C-Terminal Histidine Tag (Insect Select Expression System) A previous construct containing the gene for GnRHR (gonadotropin releasing hormone receptor) was used to make the first CXCR4 construct. The gene for GnRHR was spliced out and replaced by the isolated cDNA for CXCR4. This vector was originally the pet30a vector with the 6xHis tag at the C-terminus.

Construction of CXCR4 Construct with C-terminal FLAG tag: PCR was performed using the primers 5′ BspE1 CXCR4 and 3′ Bgl CXCR4 and engineered with unique sites for ligation of CXCR4 in frame with the FLAG tag of pFLAG-CTC (a bacterial expression vector) from Sigma. This construct is called CXCR4-FLAG-CTC. CXCR4-FLAG was then removed by digestion and filled in with Klenow fragment. The fragment containing CXCR4-FLAG was ligated into pBluebac 4.5 that was first digested then blunted with Klenow. This final construct is called CXCR4-FLAG. The construct was confirmed by restriction digestion and sequencing using standard techniques. This construct has been used for expression and has been determined to be expressed sufficiently and in active form for use in affinity purification screening.

Construction of CXCR4 Construct with C-terminal GST tag: The newly constructed CXCR4-FLAG cDNA was removed from the CTC vector and subcloned into another construct, CCR5-GST, in place of the CCR5 (using Bgl and BspE1). This created the vector for CXCR4-GST using one step. The construct was confirmed by restriction digestion and sequencing using standard techniques. This construct has been used for expression and been determined to be expressed sufficiently and in active form for use in affinity purification screening.

Construction of CXCR4 with N-terminal 6xHis tag: This construct was prepared by subcloning the CXCR4 into the commercially available vector, pBluebacHis2b (Invitrogen). The construct was confirmed by restriction digestion and sequencing using standard techniques.

The vectors for the three new constructs, CXCR4-FLAG, CXCR4-GST, and CXCR4-HIS, were used to co-transfect Sf9 cells for the production of a viral stock of each. These viral stocks were purified using a standard plaque assay and then used in experiments to infect for the optimization of expression of CXCR4 with its various C-terminal tags. High Five cells (Invitrogen) were also transfected with these CXCR4 tagged constructs and tested for expression of CXCR4. All constructs were determined to express the appropriately tagged receptor. Expression levels after 72 hours were as much as 5 times greater in High Five cells than those for Sf9 cells.

2. Activity of CXCR4

Each of the tagged CXCR4 genes (CXCR4-FLAG, CXCR4-GST, and CXCR4-HIS) were used to co-transfect Sf9 and High Five cells, as described herein. Whole cells from Sf9 and High five cell lines were lysed using hypotonic buffers (10 mM Tris, pH 7.4, 5 mM EDTA), and membrane preparations were made by homogenization and centrifugation using standard techniques known to those skilled in the art. Membrane preparations for CXCR4-GST, CXCR4-FLAG, and CXCR4-HIS were assayed using a standard radioligand binding assay. The radioligand [¹²⁵I]-SDF-1 (NEN) was incubated with membranes at room temperature for 1 hour with and without cold SDF-1 (a competing natural ligand), filtered, washed, and radioactive counts bound were detected using scintillation counting. Uninfected cells were used as a control for this experiment. The activity of the membrane preparations resulted in at least 20% active protein.

3. Solubilization of CXCR4

Both lysed whole cells and membrane preparations have been used for solubilization. Solubilization of the tagged versions of CXCR4 (CXCR4-FLAG, CXCR4-GST, and CXCR4-HIS) have been performed using many different combinations of detergents (such as, for example, NP-40, Triton X-100, β-D-maltoside, n-octylglucoside, CYMAL, Zwittergents, Tween-20, lysophosphatidyl choline, CHAPS, etc.,) salts (such as, for example, NaCl, CaCl₂, MgCl₂, MnCl₂, KCl, etc.,) buffers (such as, for example, Tris, Hepes, Hepps, Pipes, Mes, Mops, acetate, phosphate, imidazole, etc.,) at pH's ranging from about 6.8 to about 8.2. Conditions for optimal solubilization were found using Zwittergent 3-14 and low salt, e.g., low magnesium and calcium, but no NaCl (0.0 nM NaCl) at pH 8.1. In a preferred embodiment, at least 20% of the solubilized, immobilized protein is active. In highly preferred embodiments, at least 30%, 40%, 50% and 75% of the solubilized, immobilized protein is active.

4. Immobilization of CXCR4

After solubilization, CXCR4-GST was immobilized onto affinity columns for purification and as active protein ready for use. CXCR4-GST was bound and immobilized onto glutathione-agarose (Pierce) and glutathione-sepharose (Amersham Pharmacia Biotech). The immobilization of the protein in active form required the use of detergents and appropriate pH and salt conditions to maintain activity while on the column. This activity was determined by radioactive binding using radiolabeled SDF-1 (as with the membrane assay above) and competition with cold SDF-1. Uninfected cells was used as controls for this activity, as well as the column alone. These experiments demonstrated the ability to immobilize microgram quantities of the receptor in pure form sufficient for affinity purification screening onto resin in active form.

Equivalents

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

Each of the patent documents and scientific publications disclosed hereinabove is incorporated by reference herein.

We claim:

1. A method for identifying a molecule capable of binding to a G-protein coupled receptor (GPCR) comprising the steps of:
associating a GPCR having a functional conformation with a support;
interacting a naturally-derived sample with the GPCR to bind a molecule in the sample to the GPCR, wherein the GPCR is substantially free from association with a lipid layer; and
separating the molecule from the support.

2. The method of claim 1 further comprising the step of identifying the molecule.

3. The method of claim 1 wherein the naturally-derived sample comprises a tissue extract.

4. The method of claim 1 wherein the naturally-derived sample comprises a set of at least two proteins encoded by a cDNA library.

5. The method of claim 4 wherein the cDNA library is derived from a tissue.

6. The method of claim 4 wherein the cDNA library is derived from at least one cell isolated from a multi-cellular organism.

7. The method of claim 4 further comprising the steps of associating the GPCR with a second support; interacting a subset of the proteins from the set of the at least two proteins with the GPCR to bind the molecule to the GPCR; and separating the molecule from the second support.

8. The method of claim 1 wherein the naturally-derived sample is selected from the group consisting of a tissue extract, a fraction from a tissue extract, a cell culture medium, an extract from a cell grown in a tissue culture, and a fraction from an extract from a cell grown in a tissue culture.

9. A molecule identified by the method of claim 1.

10. The method of claim 1 further comprising the step of determining the function of the molecule.

11. The method of claim 1 wherein the molecule comprises a protein.

12. A compound derived from the molecule identified by the method of claim 1.

13. The method of claim 1 further comprising the step of manufacturing a compound derived from the molecule.

14. A method for identifying a molecule capable of binding to a G-protein coupled receptor (GPCR) comprising the steps of:
associating a GPCR having a functional conformation with a support;
interacting a naturally-derived first set of molecules with the GPCR, the first set comprising a first molecule capable of binding to the GPCR;
interacting a second set of proteins with the GPCR, wherein the second set comprises a subset of the first set and comprises the first molecule; and
separating the first molecule from the support.

15. The method of claim 14 wherein the first molecule comprises a protein.

16. A method for identifying a molecule capable of binding to a G-protein coupled receptor (GPCR) comprising the steps of:
identifying a GPCR having an undefined function or an undefined natural binding compound;
selecting a naturally-derived test sample;
associating the GPCR in a functional conformation with a support;
interacting the naturally-derived test sample with the GPCR to bind a molecule in the sample to the GPCR; and
separating the molecule from the support.

17. The method of claim 16 wherein the naturally-derived test sample comprises a set of at least two proteins encoded by a cDNA library.

18. The method of claim 17 further comprising the steps of associating the GPCR with a second support; interacting a subset of the proteins from the set of the at least two proteins with the GPCR to bind the molecule to the GPCR; and separating the molecule from the second support.

19. The method of claim 16 wherein naturally-derived test sample is selected from the group consisting of a tissue extract, a fraction from a tissue extract, a cell culture medium, an extract from a cell grown in a tissue culture, and a fraction from an extract from a cell grown in a tissue culture.

20. A molecule identified by the method of claim 16.

21. The method of claim 16 further comprising the step of determining the function of the molecule.

22. A compound derived from the molecule identified by the method of claim 16.

23. The method of claim 16 further comprising the step of manufacturing a compound derived from the molecule.
